(19) United States
(12) Patent Application Publication
(10) Pub. No.: US 2025/0341439 A1
(43) Pub. Date: Nov. 6, 2025

(54) VOLATILE ORGANIC COMPOUND SENSOR FOR BATTERY FAULT DETECTION AND DEVICE CONTROL

(71) Applicant: Google LLC, Mountain View, CA (US)

(72) Inventors: Sathya Nanda Gopal Kurcharlapati, San Jose, CA (US); James Robert Lim, Mountain View, CA (US); Chintan Trehan, San Jose, CA (US); Fang Liu, Palo Alto, CA (US); Chris Huang, Mountain View, CA (US)

(73) Assignee: Google LLC, Mountain View, CA (US)

(21) Appl. No.: 18/656,272

(22) Filed: May 6, 2024

(51) Int. Cl. G01M 3/16; G01N 27/12; G01N 33/00; H01M 10/42

(52) U.S. Cl. CPC G01M 3/16; G01N 27/12; G01N 33/0027; H01M 10/4228

(57) ABSTRACT

Readings may be received from a chemical sensor disposed proximate to a battery within a housing of a smart-home device. The readings from the chemical sensor may be processed to determine whether a chemical associated with an internal environment of the battery is present inside the smart-home device. A determination may then be made as to whether the battery is damaged based on whether the chemical is present inside the smart-home device. Alternatively, the device may determine that the chemical originates from outside of the housing of the device. A sequence of mitigation actions may then be executed to remedy the faulty battery or improve the indoor air quality.

### TECHNICAL FIELD

This disclosure generally describes smart home devices utilizing chemical sensors. More specifically, this disclosure describes using internal chemical sensor to distinguish between chemicals originating from a faulty internal battery and chemicals urging any outside of the device.

### BACKGROUND

Systems for remotely operating air handling systems (such as heating, ventilation, and air conditioning, or HVAC, systems) have become prevalent. In such systems, control of the air handling systems is often effectuated based on an end user's interactions with a control application that is executing on the end user's electronic device. Cloud-based servers often facilitate communication between these electronic devices and the air handling systems. While remote control of air handling systems is convenient, it may be desirable to provide a feature-rich means to effectuate local control of these air handling systems. Control devices, such as thermostats, may include a variety of sensors that may be used for monitoring environmental conditions within the home. These environmental conditions may include temperature, sunlight, noise, user presence, and/or airflow. However, existing control devices typically do not include technologies that may be employed to monitor and improve indoor air quality.

### SUMMARY

In some embodiments, a thermostat ma include a housing, a battery disposed within the housing and exposed to an internal environment of the thermostat, and a Volatile Organic Compound (VOC) sensor disposed within the housing exposed to the internal environment of the thermostat and the battery.

In some embodiments, a method of detecting battery anomalies may include receiving readings from a chemical sensor disposed proximate to a battery within a housing of a smart-home device. The method may also include processing the readings from the chemical sensor to determine whether a chemical associated with an internal environment of the battery is present inside the smart-home device. The method may further include determining whether the battery is damaged based on whether the chemical is present inside the smart-home device.

In some embodiments, a method of detecting a Volatile Organic Compounds (VOCs) in smart-home devices may include determining, using a VOC sensor of a smart-home device, that a VOC is present in a housing of the smart-home device. The smart-home device may be one of a plurality of smart-home devices present in a building, and the plurality of smart-home devices may be communicatively connected through at least one wireless network in the building. The method may also include determining, by a processor, a type of the VOC present in the smart-home device. The type of the VOC may indicate whether the VOC likely originated inside the housing of the smart-home device or outside of the housing of the smart-home device. The method may additionally include causing a sequence of one or more mitigation actions to be performed by the plurality of smart-home devices in response to determining the type of the VOC.

In any embodiments, any and all of the following features may be implemented in any combination and without limitation. The VOC sensor may be disposed within 3 cm of the battery. The VOC sensor may be sealed from an environment that is external to the housing of the thermostat such that the VOC sensor is not exposed to the environment that is external to the housing of the thermostat or to VOCs originating outside of the housing of the thermostat. Alternatively, the VOC sensor may also be exposed to an environment that is external to the housing of the thermostat such that the VOC sensor is exposed to VOCs originating outside of the housing of the thermostat. The VOC sensor may be configured to detect an electrolyte that is emitted from the battery when the battery is damaged. The battery may include a lithium-ion battery, and the electrolyte comprises one or more of EC(C3H4O3), PC(C4H6O3), DEC(C5H10O3), DMC(C3H6O3), and/or EMC(C4H8O3). The thermostat may include a processor, where the VOC sensor may generate measurements from a chemical resistance circuit, and the VOC may include a serial interface to transmit the chemical resistance circuit to the processor. The readings from the chemical sensor may include a sliding window of a plurality of readings over a time interval, and determining whether the chemical is present include calculating a statistic summarizing the plurality of readings over the time interval. A low-pass filter may be applied to the plurality of readings over the time interval. The plurality of readings may be reduced to a fewer number of readings to remove chemical resistance readings with above a threshold level of noise. The readings may include chemical resistance measurements from a Volatile Organic Compound (VOC) sensor. The readings from the chemical sensor may be provided to a neural network, where the neural network may include inputs corresponding to a plurality of the readings, at least one hidden internal layer, and outputs corresponding to specific chemical compounds. The outputs may provide a probability indicating a likelihood of the corresponding specific chemical compounds being present. The outputs may classify the specific chemical compounds as being associated with the battery or being associated with an environment external to the smart-home device. The readings may be received from the chemical sensor with a sampling period of between every 1 second and every 5 seconds. The sequence of one or more mitigation actions may include causing a ventilation system of the building to be activated when the type of VOC indicates that the VOC likely originated outside of the housing of the smart-home device. The sequence of one or more mitigation actions may include connecting to a smart-home app or server of a manufacturer of the smart-home device to arrange for a replacement or repair of the smart-home device. The sequence of one or more mitigation actions may include evaluating an outdoor air quality before causing a window of the building to be opened.

### BRIEF DESCRIPTION OF THE DRAWINGS

A further understanding of the nature and advantages of various embodiments may be realized by reference to the remaining portions of the specification and the drawings, wherein like reference numerals are used throughout the several drawings to refer to similar components. In some instances, a sub-label is associated with a reference numeral to denote one of multiple similar components. When reference is made to a reference numeral without specification to an existing sub-label, it is intended to refer to all such multiple similar components.

FIG. 1 is a block diagram of an embodiment of a smart thermostat system.

FIG. 2A is an isometric view of an embodiment of a smart thermostat.

FIG. 2B is a front view of an embodiment of smart thermostat.

FIG. 2C is a side view of an embodiment of a smart thermostat.

FIG. 3 is an exploded front isometric view of an embodiment of smart thermostat.

FIG. 4 is an exploded rear isometric view of smart thermostat.

FIG. 5A illustrates a front view of a smart thermostat backplate.

FIG. 5B illustrates a side view of a smart thermostat backplate.

FIG. 5C is an exploded front isometric view of the smart thermostat backplate.

FIG. 6 is an exploded front view of various embodiments of lens assembly.

FIG. 7 is a cross section of an embodiment of smart thermostat.

FIG. 8 is an enlarged cross section of a side view of a smart thermostat.

FIG. 9 is clip for use with a smart thermostat.

FIG. 10 is an isometric cross section of a side view of a smart thermostat.

FIG. 11A illustrates a view of a smart thermostat that includes a chemical sensor disposed within the housing of the thermostat, according to some embodiments.

FIG. 11B illustrates a side view of the thermostat where the chemical sensor may also be exposed to an external environment of the thermostat, according some embodiments.

FIG. 12 illustrates a flowchart of a method for detecting battery anomalies, according to some embodiments.

FIG. 13 illustrates a processor in communication with a chemical sensor, according to some embodiments.

FIG. 14 illustrates a diagram of a neural network used to distinguish between VOCs, according to some embodiments.

FIG. 15A illustrates a graph of a response of the chemical sensor for an external VOC, according to some embodiments.

FIG. 15B illustrates a graph of the response of the chemical sensor for a VOC from a leaking battery.

FIG. 15C illustrates a graph of responses for different gases that may be present from a defective battery, according to some embodiments.

FIG. 16 illustrates a flowchart of various operations that may be performed by the smart-home system to confirm and/or mitigate the presence of the VOC, according to some embodiments.

FIG. 17 illustrates an example smart home environment.

### DETAILED DESCRIPTION

Thermostats that communicate via a network and allow end users to interact with a heating, ventilation, and air conditioning system (referred to herein as “HVAC system,” “HVAC systems,” “air handling system,” and “air management system”) from remote locations have become prevalent. Typically, an end user will use a control application that is executing on an electronic device such as a mobile phone to connect with and operate the thermostat and/or HVAC system. Such thermostats often include advanced features such as Internet or Wi-Fi connectivity, occupancy detection, home/away/vacation modes, indoor climate sensing, outdoor climate sensing, notifications, display of current weather conditions, learning modes, and others. Thermostats such as the foregoing and others can be referred to as smart thermostats.

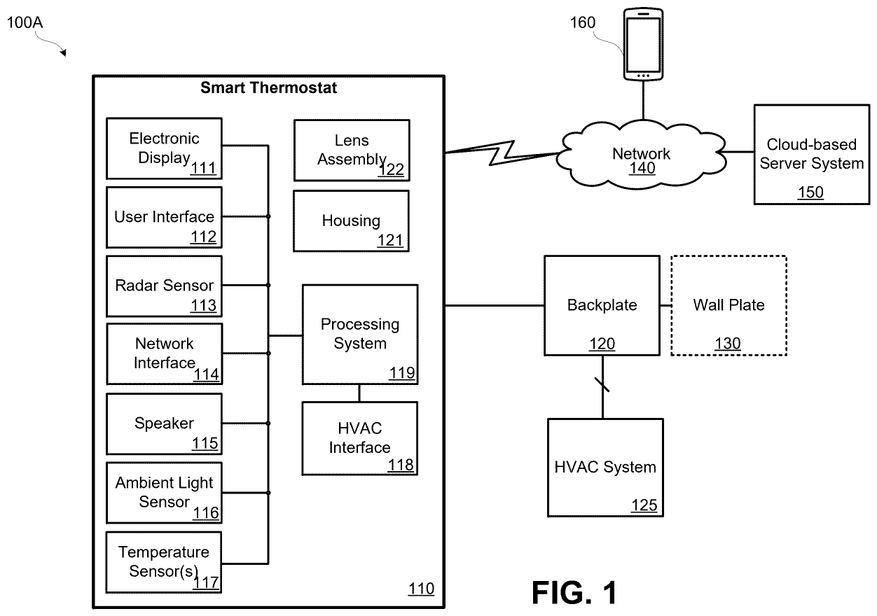

FIG. 1 is a block diagram of an embodiment of a smart thermostat system. Smart thermostat system 100A can include smart thermostat 110; backplate 120; HVAC system 12; wall plate 130; network 140; cloud-based server system 150; and computerized device 160. Smart thermostat 110 represents embodiments of thermostats detailed herein. Smart thermostat 110 can include: electronic display 111; user interface 112; radar sensor 113; network interface 114; speaker 115; ambient light sensor 116; one or more temperature sensors 117; HVAC interface 118; processing system 119; housing 121; and lens assembly 122.

Electronic display 111 may be visible through the lens assembly 122. In some embodiments, electronic display 111 is only visible when electronic display 111 is at least partially illuminated. In some embodiments, electronic display 111 is not a touch screen which can allow the electronic display 111 to serve as a user interface to receive input. If a touch sensor, the electronic display 111 may allow one or more gestures, including tap and swipe gestures, to be detected.

User interface 112 can be various forms of input devices through which a user can provide input to smart thermostat 110. In some embodiments herein, an outer rotatable ring is present as part of user interface 112. The ring can be rotated by a user clockwise and counterclockwise in order to provide input. The ring can be infinitely rotatable in either direction, thus allowing a user to scroll or otherwise navigate user interface menus. The ring (and, possibly, lens assembly 122) can be pressed inward (toward the rear of smart thermostat 110) to function as a “click” or to make a selection. The outer rotatable ring can, for example, allow the user to make temperature target adjustments. By rotating the outer ring clockwise, the target temperature can be increased, and by rotating the outer ring counterclockwise, the target temperature can be decreased. As another example, the ring can be rotated to highlight displayed icons; an inward click can be provided by a user to select a particular icon.

Radar sensor 113 may be a single integrated circuit (IC) that can emit radio waves, receive reflected radio waves, and output radar data indicative of the received reflected radio waves. Radar sensor 113 may be configured to output radio waves into the ambient environment in front of electronic display 111 of the smart thermostat 110. The radar sensor 113 may emit radio waves and receive reflected radio waves through the lens assembly 122. The radar sensor 113 may include one or more antennas, one or more radio frequency (RF) emitters, and one or more RF receivers. The radar sensor 113 may be configured to operate as frequency-modulated continuous wave (FMCW) radar. The radar sensor 113 may emit chirps of radar that sweep from a first frequency to a second frequency (e.g., in the form of a saw tooth waveform). Using receive-side beam-steering (e.g., using multiple receiving antennas), certain regions may be targeted for sensing the presence of objects and/or people. The output of the radar sensor 113, which can be a radar data stream, may be analyzed using the processing system 119. The radar sensor 113 and the processing system 119 may be referred to hereinafter as radar subsystem.

Network interface 114 may be used to communicate with one or more wired or wireless networks. Network interface 114 may communicate with a wireless local area network, such as a Wi-Fi network. Additional or alternative network interfaces may also be present. For example, smart thermostat 110 may be able to communicate with a user device directly, such as using Bluetooth or some other device-to-device short-range wireless communication protocol. Smart thermostat 110 may be able to communicate via a mesh network with various other home automation devices such as using Thread or Matter. Mesh networks may use relatively less power compared to wireless local area network-based communication, such as Wi-Fi. In some embodiments, smart thermostat 110 can serve as an edge router that translates communications between a mesh network and a wireless local area network, such as a Wi-Fi network. In some embodiments, a wired network interface may be present, such as to allow communication with a local area network (LAN). One or more direct wireless communication interfaces may also be present, such as to enable direct communication with a remote temperature sensor installed in a different housing external and distinct from housing 121. The evolution of wireless communication to fifth generation (5G) and sixth generation (6G) standards and technologies provides greater throughput with lower latency which enhances mobile broadband services. 5G and 6G technologies also provide new classes of services, over control and data channels, for vehicular networking (V2X), fixed wireless broadband, and the Internet of Things (IoT). Smart thermostat 110 may include one or more wireless interfaces that can communicate using 5G and/or 6G networks.

Speaker 115 can be used to output audio. Speaker 115 may be used to output beeps, clicks, synthesized speech, or other audible sounds, such as in response to the detection of user input via user interface 112.

Ambient light sensor 116 may sense the amount of light present in the environment of smart thermostat 110. Measurements made by ambient light sensor 116 may be used to adjust the brightness of electronic display 111. In some embodiments, ambient light sensor 116 senses an amount of ambient light through lens assembly 122. Therefore, compensation for the reflectivity of lens assembly 122 may be made such that the ambient light levels are correctly determined via ambient light sensor 116. In some implementations, a light pipe is present between ambient light sensor 116 and lens assembly 122 such that, in a particular region of lens assembly 122, light that is transmitted through lens assembly 122, is directed to ambient light sensor 116, which may be mounted to a printed circuit board (PCB), such as a PCB to which processing system 119 is attached.

One or more temperature sensors 117, may be present within smart thermostat 110. The one or more temperature sensors 117 may be used to measure the ambient temperature in the environment of smart thermostat 110. One or more additional temperature sensors that are remote from smart thermostat 110 may additionally or alternatively be used to measure the temperature of the ambient environment.

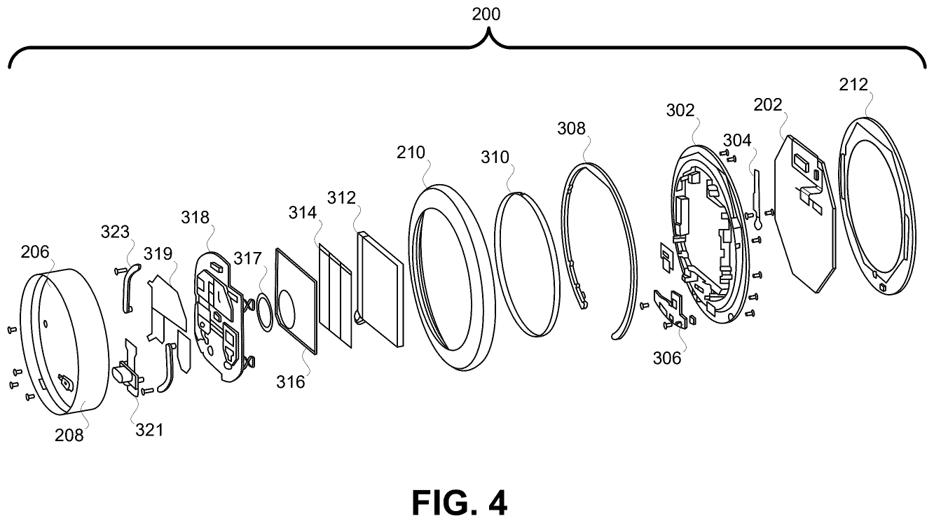

Lens assembly 122 may have a transmissivity sufficient to allow illuminated portions of electronic display 111 to be viewed through lens assembly 122 from an exterior of smart thermostat 110 by a user. Lens assembly 122 may have a reflectivity sufficient such that portions of lens assembly 122 that are not illuminated from behind appear to have a mirrored effect to a user viewing a front of smart thermostat 110. Further detail regarding the lens assembly 122 are provided in relation to FIGS. 4-7.

HVAC interface 118 can include one or more interfaces that control whether a circuit involving various HVAC control wires that are connected either directly with smart thermostat 110 or with backplate 120 is completed. A heating system (e.g., furnace, boiler, heat pump), cooling system (e.g., air conditioner, heat pump), fan, or some combination thereof may be controlled via HVAC wires by opening and closing circuits that include the HVAC control wires. In some installations, one a heating system or cooling system is controlled by the smart thermostat 110; in other embodiments, the smart thermostat 110 may control both a heating system and a cooling system.

Processing system 119 can include one or more processors. Processing system 119 may include one or more special-purpose or general-purpose processors. Such special-purpose processors may include processors that are specifically designed to perform the functions detailed herein. Such special-purpose processors may be ASICs or FPGAs which are general-purpose components that are physically and electrically configured to perform the functions detailed herein. Such general-purpose processors may execute special-purpose software that is stored using one or more non-transitory processor-readable mediums, such as random access memory (RAM), flash memory, a hard disk drive (HDD), or a solid state drive (SSD) of smart thermostat 110.

Processing system 119 may output information for presentation to electronic display 111. Processing system 119 can receive information from the one or more temperature sensors 117, user interface 112, radar sensor 113, network interface 114, and ambient light sensor 116. Processing system 119 can perform bidirectional communication with network interface 114. Processing system 119 can output information to be output as sound to speaker 115. Processing system 119 can control the HVAC system 125 via HVAC interface 118.

Housing 121 may house and/or attach with all of the components of smart thermostat 110, either directly or via other components. For example, lens assembly 122 may adhere to the electronic display 111, which is attached with housing 121.

The smart thermostat 110 may be attached (and removed) from backplate 120. Some number of HVAC control wires may be attached with terminals or receptacles of backplate 120. Such HVAC control wires electrically connect backplate 120 with the HVAC system 125, which can include a heating system, cooling system, ventilation system, or some combination thereof. Backplate 120 can allow the smart thermostat 110 to be attached and removed from backplate 120 without affecting the electronic connections of the HVAC control wires with backplate 120. In other embodiments, such control wires are directly connected with smart thermostat 110. In some embodiments, wall plate 130 may additionally be installed between backplate 120 and a surface, such as a wall, such as for aesthetic reasons (e.g., cover an unsightly hole through which HVAC wires protrude from the wall).

Network 140 can include one or more wireless networks, wired networks, public networks, private networks, and/or mesh networks. A home wireless local area network (e.g., a Wi-Fi network) may be part of network 140. Network 140 can include the Internet. Network 140 can include a mesh network, which may include one or more other smart home devices, may be used to enable smart thermostat 110 to communicate with another network, such as a Wi-Fi network. Smart thermostat 110 may function as an edge router that translates communications from a relatively low power mesh network received from other devices to another form of network, such as a relatively higher power network, such as a Wi-Fi network.

Cloud-based server system 150 can maintain an account mapped to smart thermostat 110. Smart thermostat 110 may periodically or intermittently communicate with cloud-based server system 150 to determine whether setpoint or schedule changes have been made. A user may interact with smart thermostat 110 via computerized device 160, which may be a mobile device, smartphone, tablet computer, laptop computer, desktop computer, or some other form of computerized device that can communicate with cloud-based server system 150 via network 140 or can communicate directly with smart thermostat 110 (e.g., via Bluetooth or some other device-to-device communication protocol). A user can interact with an application executed on computerized device 160 to control or interact with smart thermostat 110.

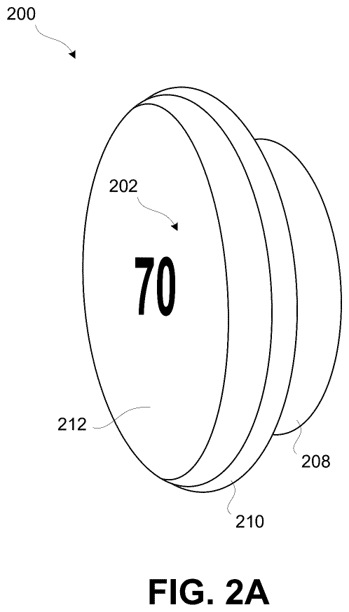

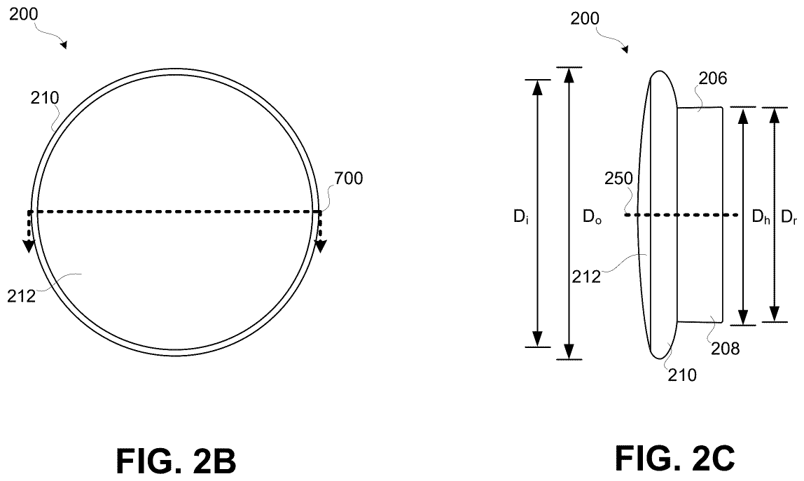

FIG. 2A is an isometric view of an embodiment of a smart thermostat 200. Smart thermostat 200 can represent an embodiment of smart thermostat 110 of FIG. 1. In FIG. 2A, electronic display 202, located behind lens assembly 212, is active in displaying a setpoint temperature. The housing of smart thermostat 200 can define sidewall 208. Sidewall 208 may be generally cylindrical according to various embodiments. A diameter of the sidewall 208 may be smaller than a diameter of the electronic display 202 and ring 210 according to various embodiments and as illustrated in FIG. 2A. Ring 210 can function as detailed in relation to user interface 112. Either attached with housing 121 or attached with components connected with housing 121 is lens assembly 212. Lens assembly 212 may include a reflective layer having a reflectivity such that when the electronic display 202 is not illuminated, lens assembly 212 appears to be a mirror when viewed by a user.

In some embodiments, ring 210 is mounted to lens assembly 212. In other embodiments, ring 210 can be rotated clockwise and counterclockwise independent of lens assembly 212. In some embodiments, housing 121 includes a display frame (not visible in this view) that further supports electronic display 202 and lens assembly 212.

Electronic display 202 is housed behind lens assembly 212 such that, when illuminated, the portion of electronic display 202 that is illuminated is visible through lens assembly 212 by a user. In some embodiments, due to the reflectivity of lens assembly 212, an edge of electronic display 202 is not visible to a user regardless of whether electronic display 202 is illuminated, partially illuminated, or not illuminated. Therefore, the overall effect experienced by a user may be that lens assembly 212 appears as a mirror and portions of electronic display 202, when illuminated, are visible through lens assembly 212.

In various embodiments, around an axis perpendicular to the display face of electronic display 202, the ring 210 has an inner diameter and an outer diameter and both the inner diameter and the outer diameter of ring 210 are larger than a diameter of sidewall 208 of housing 121.

FIG. 2B is a front view of an embodiment of smart thermostat 200. When mounted on a wall or other surface, lens assembly 212 is opposite the portion of smart thermostat 200 that mounts to the wall or other surface. Therefore, when a user is facing mounted smart thermostat 200, lens assembly 212 is visible. Lens assembly 212 can form an uninterrupted circular surface with no gaps, holes, lens, or other discontinuities present on the outermost surface of lens assembly 212. Lens assembly 212 has sufficient transmissivity to allow light emitted by electronic display 202 located within housing 206 to be visible through lens assembly 212. Further, lens assembly 212 may have sufficient reflectivity such that a mirrored effect is present on portions of lens assembly 212 that are not currently being illuminated from behind by electronic display 202.

FIG. 2C is a side view of an embodiment of a smart thermostat. When smart thermostat 200 is mounted to a wall or other surface, sidewall 208 of housing 121 is visible. Around an axis 250, the ring 210 has an inner diameter Di and an outer diameter Do and both the inner diameter Di and the outer diameter Do of the ring 210 are larger than a diameter Dh of sidewall 208 of housing 121. According to various embodiments, sidewall 208 of housing 121 can be generally cylindrical and can have a consistent diameter along a length thereof. Alternatively, a diameter of sidewall 208 can increase as a distance from lens assembly 212 increase.

In some embodiments, ring 210 has a smallest diameter at the rearmost portion of ring 210. Dr is indicative of the diameter of ring 210 where ring 210 meets sidewall 208. This arrangement can help facilitate a user's fingers reaching around ring 210, grasping ring 210, and rotating in either direction. In some embodiments, along axis 250, sidewall 208 may have a diameter of approximately Dr wherein ring 210 and sidewall 208 meet. In some embodiments, the diameter of sidewall 208 can increase as the distance from ring 210 increases.

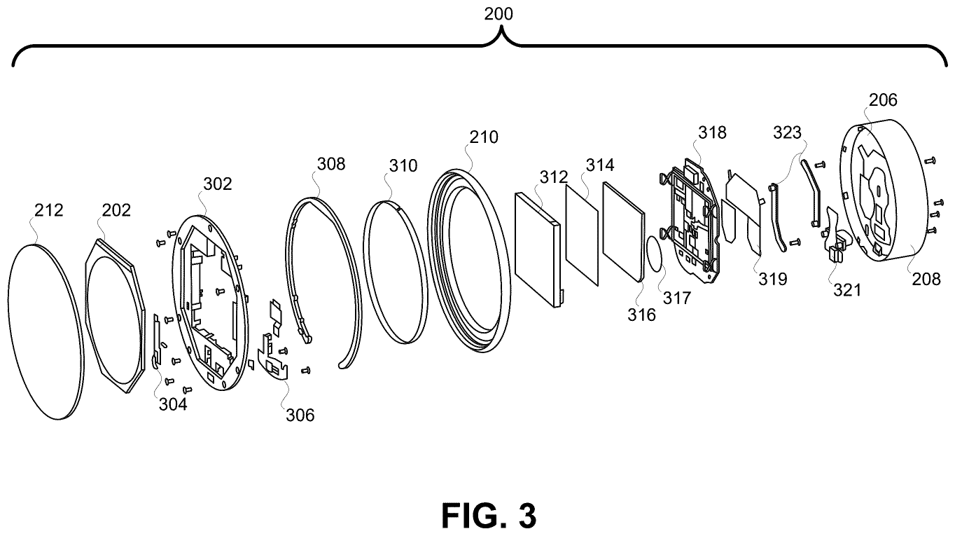

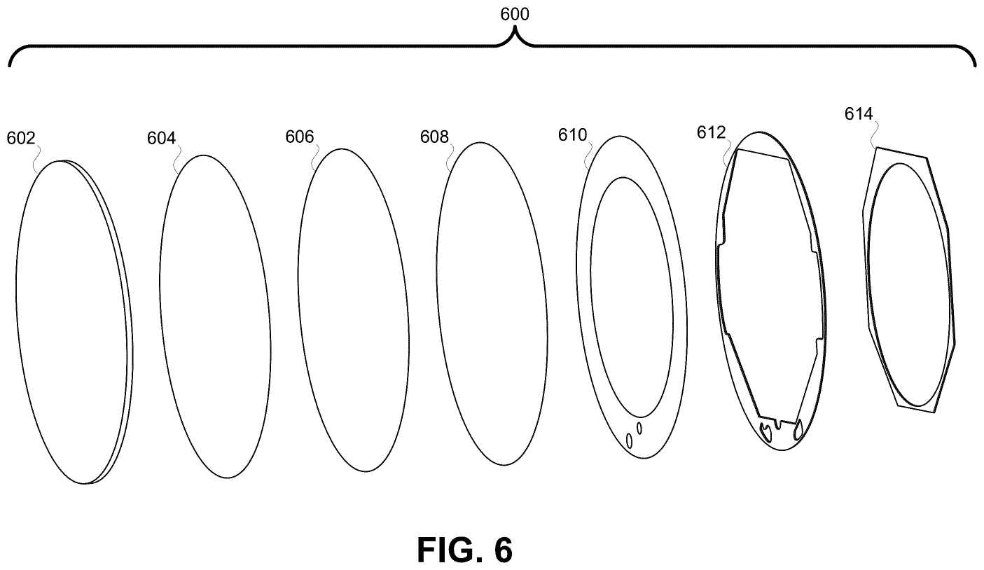

FIG. 3 is an exploded front isometric view of an embodiment of smart thermostat 200. FIG. 4 is an exploded rear isometric view of smart thermostat 200. Viewing the components of the smart thermostat 200 left to right, lens assembly 212 forms an outermost domed surface of smart thermostat 200. Adjacent lens assembly 212 may be electronic display 202. Electronic display 202 may be a liquid-crystal display (LCD) or organic light emitting diode (OLED) display according to various embodiments. In at least some embodiments, one or more adhesives may be used to attach electronic display 202 with lens assembly 212. An exploded view of lens assembly 212 is provided in relation to FIG. 6.

According to at least some embodiments, electronic display 202 is supported by a display frame 302. Smart thermostat 200 further includes one or more antenna assemblies 304 for communicating with a network and/or other electronic devices. Antenna assembly 304 can be used for communicating with wireless local area networks (e.g., Wi-Fi), device-to-device communication (e.g., Bluetooth), and/or communicating with mesh networks (e.g., Thread). Smart thermostat 200 includes one or more sensor boards, such as sensor daughterboard 306. One or more temperature sensors may be installed on sensor daughterboard 306. Use of sensor daughterboard 306 can help isolate the one or more temperature sensors from heat generated by other components.

Smart thermostat 200 may further include clip 308 for coupling ring 210 and display frame 302 supporting electronic display 202. Clip 308 may act as an axial constraint for smart thermostat 200. In particular, clip 308 prevents electronic display 202, display frame 302, and ring 210 from decoupling from one another in the assembled configuration.

As shown in FIGS. 3-4, smart thermostat can include magnetic strip 310. According to various embodiments, ring 210 rotates relative to sidewall 208 of housing 206 and a backplate when smart thermostat 200 is mounted to a surface. In various embodiments, a sensor installed on a sensor board, such as sensor board 306 and magnetic strip 310 are used for detecting rotation of the ring 210 during use.

According to various embodiments, ring 210 is mounted to housing 206 such that ring 210 can be rotated clockwise and counterclockwise. Ring 210 may include polished stainless steel and a finish applied using physical vapor deposition (PVD). Ring 210 further advantageously provides an aesthetic appearance as the finish of the ring 210 appears seamless relative to lens assembly 212 having a mirrored effect.

Further internal components of smart thermostat 200 include battery 312 and battery adhesive 314. Battery 312 can be a secondary battery and can provide power to the various components of smart thermostat 200, including electronic display 202 and processing system 119. Battery adhesive 314 may be used to adhere battery 312 within housing 206 although the battery 312 (or any other components of the smart thermostat 200) may be secured within the housing 206 using other means. For example, various components may be secured using adhesives, screws, wires, clips, or the like.

Smart thermostat 200 includes processing system 316. According to some embodiments, processing system 316 is a system-on-a-chip (SoC) including various processing parts, memory, modems, etc. Processing system 316 may be in electric communication with one or more antennas present on antenna assembly 304, sensor board 306, electronic display 202, etc., for performing various functions of the smart thermostat 200 and outputting results based on user input (e.g., in response to the user rotating the ring 210 and/or user input via an external mobile device). Adjacent processing system 316 may be piezo sensor 317. Additional components of the processing system 316 or components that work with processing system 316 are also shown in FIGS. 3-4. For example, multi-layer board (MLB) 318 may be provided for performing various functions of smart thermostat 200, in a manner that would be appreciated by one having ordinary skill in the art. In some embodiments, MLB 318 may include a Universal Serial Bus (USB) port for electrically coupling smart thermostat 200 to another electronic device for various updates, servicing, or the like. Various springs 319 for supporting components, flexes 321 for enabling flexible and high-density interconnects between printed circuit boards (PCBs), LCDs, etc., and additional links 323 may also be included in the internal components of smart thermostat 200.

Smart thermostat 200 may include more or fewer components than those shown in FIGS. 3-4. In various embodiments, the components may be in one or more configurations other than the configuration shown in FIGS. 3-4. Advantageously, various components of smart thermostat 200 are optimized to be condensed into housing 206 such that the overall side profile of smart thermostat 200 is significantly thinner than a side profile of other commercially available smart thermostats.

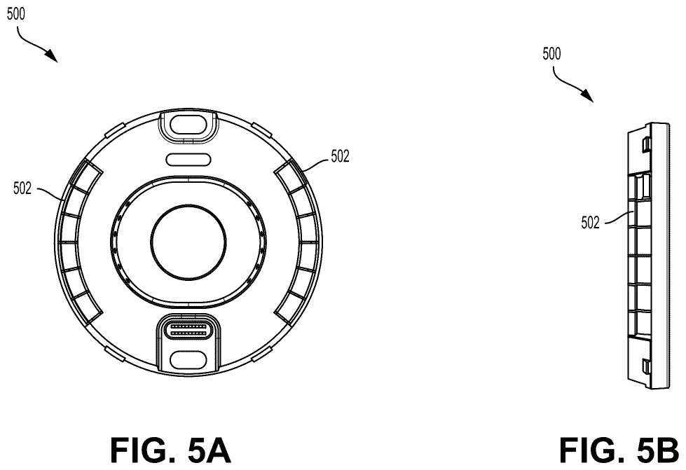

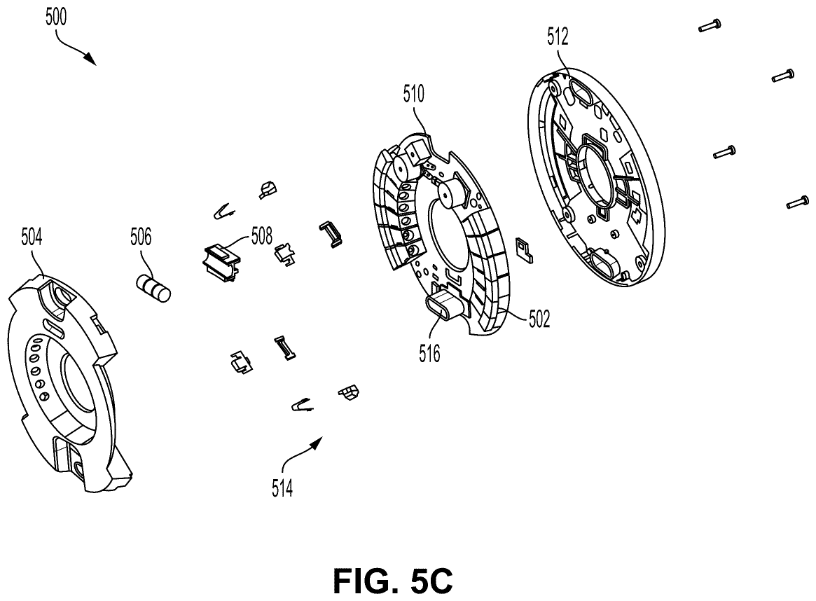

FIGS. 5A-5B illustrate a front view and a side view of a smart thermostat backplate. According to various embodiments, an electronic device, such as smart thermostat 200 described in detail above, may be mounted to a wall or other surface by a backplate 500. The backplate 500 may include a plurality of wire terminals 502 for receiving wires that are connected with a heating, ventilation, and cooling (HVAC) system. For example, the backplate 500 may include multiple receptacles, with each receptacle designated to receive a particular HVAC control wire. Backplate 500 can define one or more holes configured to receive fasteners or the like for securing backplate 500 and, if being used, a trim plate or the like, to a surface, such as a wall. The backplate 500 can removably attached with the thermostat housing, such as thermostat housing 206 described above.

In some embodiments, a smart thermostat may be attached (and removed) from backplate 500. HVAC control wires may be attached with terminals or receptacles of backplate 500. Alternatively, such control wires may be directly connected with the smart thermostat. In some embodiments, a trim plate may additionally be installed between the backplate 500 and a surface, such as a wall, such as for aesthetic reasons (e.g., cover an unsightly hole through which HVAC wires protrude from the wall).

FIG. 5C is an exploded front isometric view of the smart thermostat backplate of FIGS. 5A and 5B. Visible in this view, the backplate 500 includes a cap 504, a level 506, a level holder 508, and a coupling plate 510. Various components of the backplate 500 are coupled to one another with one or more fasteners 514. Fasteners 514 may be screws, nails, or some other form of fastener. Fasteners 514 can securely hold backplate 500 and, possibly, a trim plate (not shown) to a surface, such as a wall. A thermostat may removably attach with backplate 500. A user may be able to attach thermostat to backplate 500 by pushing thermostat against backplate 500. Similarly, a user can remove the thermostat from backplate 500 by pulling the thermostat away from backplate 500. When the thermostat is connected with backplate 500, the thermostat is electrically connected various HVAC control wires that have been connected with the receptacles of backplate 500 as would be appreciated by one having ordinary skill in the art.

Further visible in FIG. 5C, a cap 504 for protecting various internal components from damage and for providing an aesthetically pleasing appearance when the electronic device is not mounted to the backplate 500. The cap 504 covers a level 506 for properly mounting the electronic device and/or the backplate 500 to a surface. For example, it would be desirable to have text displayed on the electronic display of the smart thermostat to be straight across (e.g., perpendicular to the ground, etc.). The level 506 may be a bubble level in at least some embodiments. A level holder 508 may be provided to align the level 506 relative to the cap 504, a coupling plate 510, and a base 512. Additional coupling mechanisms may be provided including adhesives, screws, snaps, wires, or the like. The coupling plate 510 may include one or more fasteners as described in detail above. The coupling plate 510 may further include a board-to-board (BTB) connector 516 in some embodiments.

The backplate 500 may include more or less components than those shown in FIGS. 5A-5C. In various embodiments, the components may be in one or more configurations other than the configuration shown in FIGS. 5A-5C. For example, the backplate 500 may be part of a greater thermostat mounting system including a trim plate, batteries, various fasteners, sensors, or the like.

FIG. 6 is an exploded front view of various embodiments of lens assembly 600. Lens assembly 600 can represent embodiments of lens assembly 122 and 212. In particular, FIG. 6 illustrates an embodiment of a stack of components that can be used to create lens assembly 122. Lens assembly 600 can include: domed lens 602; optically clear adhesive (OCA) layer 604; tinted ink layer 606; mirror film 608; masking layer 610; frame pressure sensitive adhesive (PSA) 612; and display PSA 614. While embodiments of lens assembly 600 may be used on smart thermostat 200, embodiments of such a lens assembly may be used on other forms of smart devices. For instance, lens assembly 600 can be incorporated as part of a smart assistant device or a smart watch.

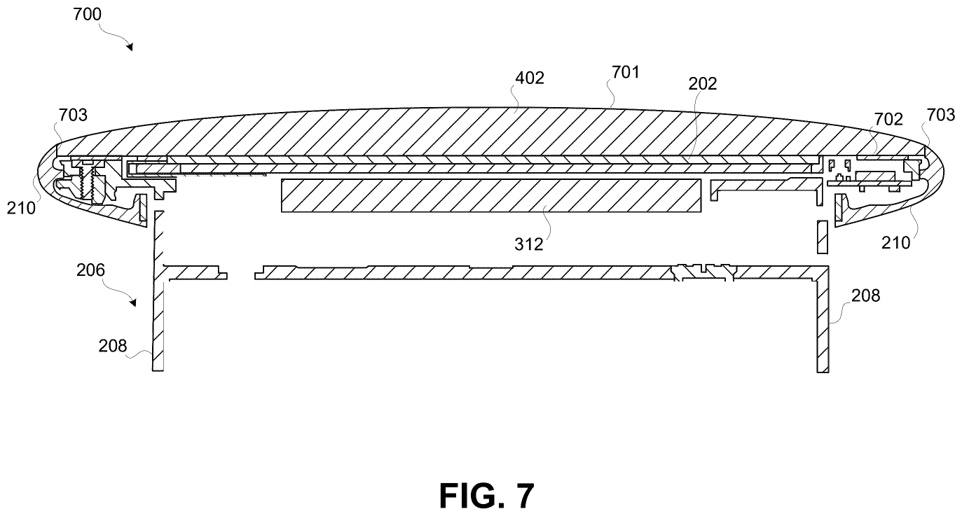

Domed lens 602 may be domed on an outer surface and flat on an inner surface that is in contact with OCA lay 604. Further detail regarding the shape of domed lens 602 is provided in reference to FIG. 7. Domed lens 602 can be formed from polymethyl methacrylate (PMMA), which can provide a transparency similar to glass. Other plastic or acrylic materials are also possible. Domed lens 602 may also be formed from glass. Domed lens 602 can be formed using injection compression molding. Injection compression molding can be used because it allows for defect-free surfaces to be formed. To perform injection compression molding of domed lens 602, material can be injected into a nearly closed mold. The mold may then be compressed such that the injected material conforms to the shape of the mold. Excess material can be removed, such as through machining.

Domed lens 602 is circular and does not have any holes, vents, gaps, or other discontinuities present on it. Similarly, no holes, vents, gaps, or other discontinuities are present on at least OCA lay 604, tinted ink layer 606, and mirror film layer 608. Having continuous material helps to maintain a consistent visual effect across the entirety of lens assembly 600 as viewed by a user.

OCA lay 604 can be a pressure or temperature sensitive adhesive that adheres domed lens 602 with tinted ink layer 606. Tinted ink layer 606 can be a transparent layer that tints light passing through tinted ink layer 606. Since tinted ink layer 606 is closer to domed lens 602 than mirror film layer 608, both light by mirror film layer 608 and light emitted by electronic display 111 is tinted. The color used for tinting can be selected based on aesthetics.

Mirror film layer 608 may have sufficient reflectivity that when electronic display 111 is not illuminated, a user viewing lens assembly 400 may see a reflection of himself, herself, or the ambient environment. For example, mirror film layer 608 can be Toray® 125FH-40 mirror film. Mirror film layer 608 may be polarized. Due to the way some mirror films are manufactured, throughout a roll of mirror film, the direction of polarization can vary. When a piece of mirror film is stamped or cut out to form mirror film layer 608, the direction of polarization may be determined in order to orient in relation the electronic display, which also outputs polarized light. If orientation is not controlled, visibility of the electronic display through mirror film layer 608 may be adversely affected. Further detail regarding orientation of mirror film layer 608 is detailed in relation to FIG. 7.

Masking layer 610 can be used to block a user from viewing components blocked by the opaque portions of masking layer 610. Masking layer 610 may be black or another dark color to make it difficult to see through mirror film layer 608. Masking layer 610 can obscure a view of frame adhesive 612 and display adhesive 614. Masking layer 610 may be asymmetric. Therefore, it must be oriented in a particular orientation with respect to other components of smart thermostat 200. For example, masking layer 610 includes a hole for an ambient light sensor to have a field of view of the ambient environment through domed lens 602, OCA lay 604, tinted link layer 606, and mirror film layer 608.

Furthermore, the masking layer 610 may help enhance the effect that the electronic display is seamless with lens assembly 400. A color value for masking layer 610 may be selected, having an appropriate lightness value, such that it is difficult or impossible for a user to visually see an edge of the electronic display screen within the smart device. By obscuring an edge of the edge of the electronic display, a user may have the impression that the entire region behind domed lens 602 is electronic display 111.

Obscured behind masking layer 610 may be two separate adhesive layers. Frame adhesive layer 612 may adhere domed lens layer 402, OCA lay 604, tinted link layer 606, mirror film layer 608, and masking layer 610 to display frame 302. Display adhesive layer 614 may adhere domed lens layer 402, OCA lay 604, tinted link layer 606, mirror film layer 608, and masking layer 610 to electronic display 202. Different types of adhesives may be used to provide better adhesion to the material of electronic display 202 and display frame 302. Adhesive layer 612 and display adhesive layer 614 may both be different types of pressure sensitive adhesives (PSAs). In other embodiments, a single adhesive layer may be used. For example, 3M® 5126-025 may be used as the PSA.

FIG. 7 is a cross section 700 of an embodiment of smart thermostat 200. The location and direction of cross section 700 is indicated on FIG. 2B. The domed profile of domed lens 602 is visible in the cross section 700 of FIG. 7. Surface 701 is the outer surface of domed lens 602 that is adjacent the ambient environment and which a user can touch. An entirety of surface 701 is convex from edge to edge. Surface 702 is the inner surface and adheres with OCA layer 604. OCA layer 604 and other layers of lens assembly 600 are not visible in FIG. 7. An entirety of surface 702 can be flat. Surface 703 forms a circumference around the entirety of domed lens 602. Surface 703 is perpendicular or approximately perpendicular (defined as within 5° of perpendicular) to surface 702.

Electronic display 202 is disposed under the domed lens 602 and surrounded by rotatable ring 710. In particular, ring 210 surrounds surface 703 of domed lens 602 and couples to housing 206, which has a cylindrical sidewall 208.

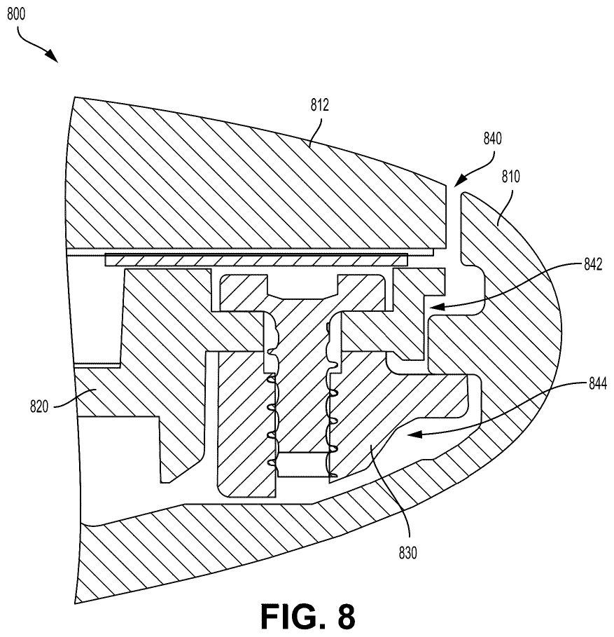

FIG. 8 is an enlarged cross section of a side view of a smart thermostat. Electronic device 800 may be similar to smart thermostat 200 and smart thermostat 500. Similar components may be similarly numbered and have similar form and function unless otherwise noted herein. As shown in FIG. 8, the clip 830, the display frame 820, and the ring 810 are assembled such that a gap 840 is formed between an outer perimeter of the domed lens 812 and a corresponding internal perimeter of the ring 810. In various embodiments, the gap 840 is not visible to the user facing the electronic device 800. For example, the mirrored reflective cover of the domed lens 812 smoothly transitions to the polished finish of the ring 810 with no disruptions. The gap 840 is optimized to be as small as possible while enabling the ring 810 to be rotated relative to the domed lens 812 and/or the electronic display (not shown in this view).

According to various embodiments, the display frame 820 includes a grease trap recess 842 for directing grease between the display frame 820 and the clip 830. For example, grease may be applied between a vertical interface (such as formed by the grease trap recess 842) of the display frame 820 and the ring 810 for continuous rotation of the ring 810 relative to the rest of the electronic device 800 (e.g., including the sidewall of the housing and the backplate) without disruption. In exemplary embodiments, a grease is applied such that the user experiences a pleasing, viscous feeling when rotating the ring 810. The grease may include a damping grease and/or a dry grease. Different types of grease may be applied at different regions between the components unless otherwise noted herein.

In at least some embodiments, the clip 830 is formed to reduce grease shearing between the clip 830 and the ring 810 at location 844. For example, grease applied at the grease trap recess 842 may be displaced to an area proximate location 844. The combination of the tuned gap 840 and grease application enhances the user experience during rotation of the ring 810 and selection of various icons and/or information displayed on the electronic display when the information is visible (e.g., when the electronic display is “ON”) through the domed lens 812.

In various embodiments, one or more temperature sensors (not shown) may be disposed between the ring 810 and the clip 830 and/or the display frame 820. For example, the one or more temperature sensors may be disposed in the portion of the electronic device 800 that overhangs the sidewall (not shown) that mounts the electronic device 800 to a mounting surface. Said another way, the electronic device 800 may form a “mushroom” shape and one or more temperature sensors are disposed proximate an outer perimeter of the “cap” of the mushroom.

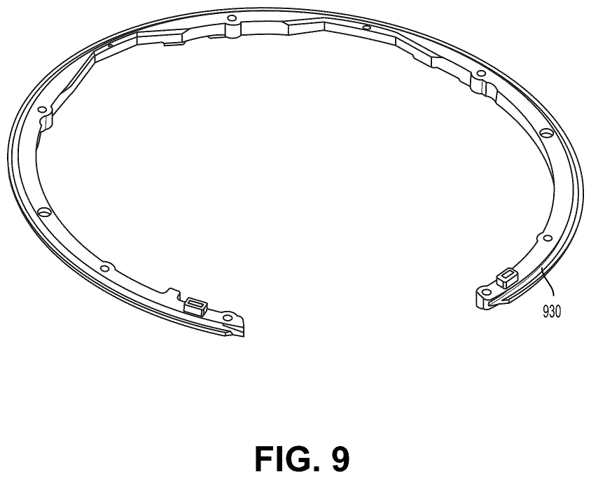

FIG. 9 is clip for use with a smart thermostat. The clip 930 may be of the same type as various clips described herein. The clip 930 may be a C-clip as shown in FIG. 9. The clip 930 acts as an axial constraint for various components of the electronic device and couples at least the display frame and the ring. The clip 930 is optimized for assembly such that the clip 930 is relatively thin within the electronic device housing. The open end of the clip 930 as shown in FIG. 9 enables efficient installation and removal of the clip 930 during servicing or other activities involving disassembling the electronic device.

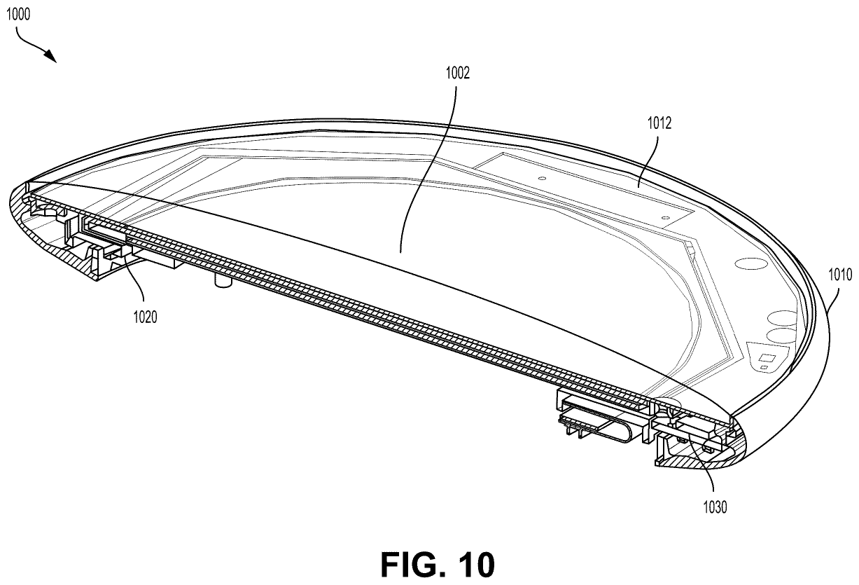

FIG. 10 is an isometric cross section of a side view of a smart thermostat. FIG. 10 provides another view of the various electronic devices described in detail above. In particular, electronic device 1000 may be similar to other electronic devices described above and similar components may be similarly numbered and have similar form and function unless otherwise noted herein. The domed profile of a domed lens 1012 is visible in the cross section of FIG. 10. An electronic display 1002 is disposed under the domed lens 1012 and supported by a ring 1010 and a display frame 1020 as described in detail above. In particular, the ring 1010 surrounds the domed lens 1012. The clip 1030 couples the display frame 1020 supporting the electronic display 1002 to the housing (not shown).

In addition to the many features and components described in relation to the smart thermostat above, some embodiments may further include specific sensors and implement specific algorithms for monitoring and improving indoor air quality. For example, indoor air quality within a home may be impacted by many different elements, including natural disasters, seasonal variations, issues with indoor air conditioning systems, local environmental factors, cleaning agents, floor finishes, home remodeling projects, and even nearby industrial sources. The smart thermostat described herein may be configured to monitor indoor air quality, detect sources of volatile chemicals, and initiate a set of mitigation actions configured to improve the indoor air quality.

In addition to air-quality issues that originate external to the thermostat, some volatile chemicals may originate from inside the thermostat. For example, the smart thermostat may be equipped with an internal power supply, such as a lithium-ion polymer battery for supplemental and/or backup power. These internal batteries may include internal chemicals, such as electrolytes, that are normally contained within the housing of the battery. However, a damaged battery may release these chemicals. This may affect the performance of the battery and possibly indicate a safety hazard or defective thermostat.

Prior to this disclosure, thermostats were unable to distinguish between volatile chemicals originating from outside of the thermostat and volatile chemicals originating from inside the thermostat. Thus, any remedial actions taken by the thermostat were not guaranteed to be effective. For example, circulating indoor air using the HVAC system would do little to remediate a situation where a pouch breach had occurred on the internal battery. This could lead to false positives, unnecessary warnings to the user, and/or unnecessary actuation of various HVAC functions, including fans, humidifiers, air purifiers, and so forth.

The embodiments described herein solve these and other technical problems by providing a chemical sensor, such as a volatile organic chemical (VOC) sensor, within the thermostat housing. This chemical sensor may be exposed to an internal environment of the thermostat that includes an internal battery. The chemical sensor may be configured to detect volatile chemicals and distinguish between types of chemicals that originate from outside of the thermostat housing versus types of chemicals that originate from inside the thermostat housing. For example, the chemical sensor may be configured to identify and classify groups of volatile chemicals that may be present as organic electrolytes used in a battery of the thermostat. When a volatile chemical is detected, the thermostat processor may receive readings from the chemical sensor and determine whether the chemical is an indication of a battery fault or indication that the indoor air quality of the home should be addressed. Depending on the classification, the thermostat may work in conjunction with a smart-home controller, such as a home app or digital home assistant, to initiate mitigation actions to address either the faulty battery or the indoor air quality. These mitigation actions initiated by the smart home system may further distinguish between internal and external chemical sources and serve to eliminate false positives and/or more effectively resolve the issue.

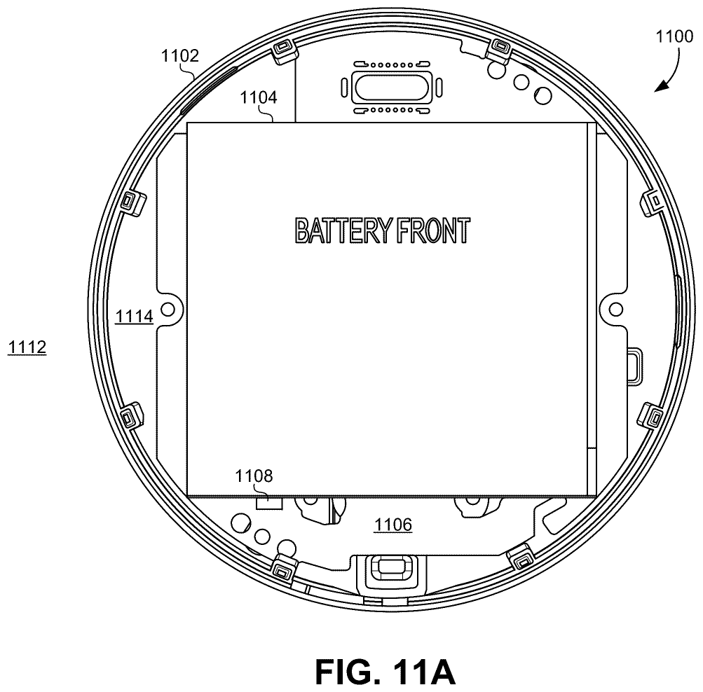

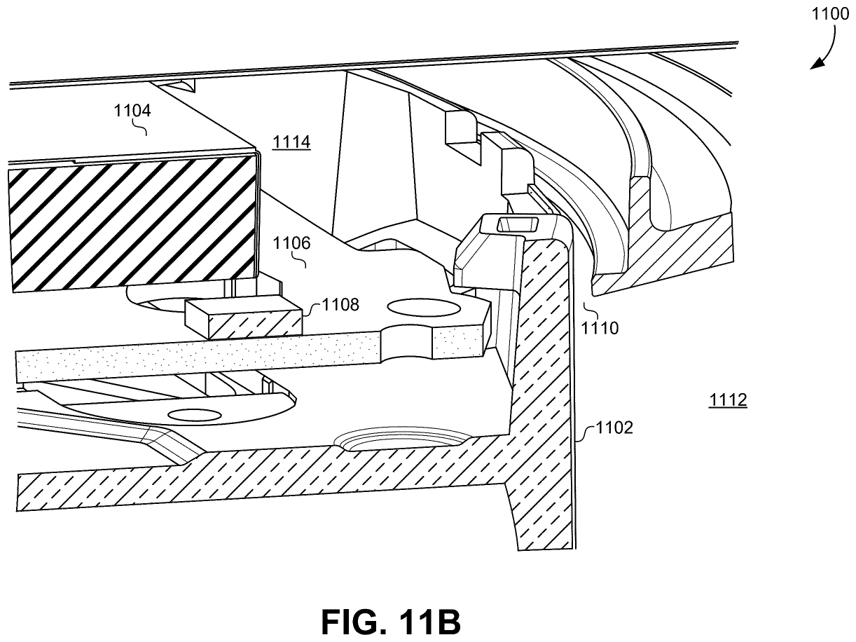

FIG. 11A illustrates a view of a smart thermostat 1100 that includes a chemical sensor disposed within the housing of the thermostat, according to some embodiments. As described above, the thermostat 1100 may include a battery 1104. The battery may include a lithium-ion battery, a lithium-ion polymer battery, and/or any other type of rechargeable or single-use battery. For example, a lithium-ion battery may include one or more electrolytes internal to the battery 1104. The battery 1104 may include a housing that seals the internal elements of the battery from the internal environment of the thermostat 1100. For example, the housing may normally seal the electrolytes inside the housing such that there is no exposure of the electrolytes to the internal environment of the thermostat. For example, the electrolytes may include one or more of EC(C3H4O3), PC(C4H6O3), DEC(C5H10O3), DMC(C3H6O3), and/or EMC(C4H8O3). The battery may also include graphite, lithium cobalt, lithium hexafluorophosphate, diethyl carbonate, propylene carbonate, ethylene carbonate, copper, aluminum, nickel, and so forth.

If the battery 1104 is damaged, the internal environment of the thermostat 1100 may be exposed to the leaking electrolytes of the battery 1104. For example, the lithium battery may include a pouch cell battery that may be susceptible to puncture. In these pouch batteries, the anodes, cathodes, and other battery components are maintained in a flexible foil pouch. While this type of battery adds a minimal amount of additional weight to the thermostat 1100, the pouch may provide relatively little protection against puncture. The battery 1104 may be damaged in a number of different ways at any time during the lifecycle of the thermostat 1100. For example, the battery may become damaged during manufacture and/or transfer of the battery prior to installation in the thermostat 1100. In some instances, the battery may be damaged when it is installed in the thermostat 1100 during a manufacturing process of the thermostat 1100. Depending on the internal configuration of some thermostats, the battery 1104 may be damaged if the thermostat is dropped or otherwise exposed to excessive shock. Additionally, repairs or disassembly by the user or even a technician may cause damage to the battery 1104. Thus, even though the battery design of the thermostat 1100 illustrated in FIG. 11A may be particularly robust and protected from damage, other thermostat designs may not protect the battery 1104 to the same extent. Additionally, unpredictable situations may arise where even the most protected battery 1104 may be susceptible to damage.

If damage to the battery 1104 occurs, the internal electrolytes and/or other volatile chemicals may be released into the internal environment of the thermostat 1100. As described above, the battery 1104 may include a housing 1102 that separates an internal environment 1114 of the thermostat 1100 from an environment 1112 that is external to the housing 1102 of the thermostat 1100. A chemical sensor 1108 may be disposed proximate to the battery 1104 within the housing 1102. For example, the chemical sensor 1108 may be mounted to a same printed circuit board (PCB) 1106 as the battery 1104. The chemical sensor 1108 may be mounted to a same side of the PCB 1106 as the battery 1104 such that any chemicals emitted from the battery 1104 may be readily detected by the chemical sensor 1108. For example, some implementations of the chemical sensor 1108 may include an opening in a top housing of the chemical sensor 1108 that exposes a chemical resistance circuit to the internal environment 1114 of the thermostat 1100. When mounted on the same side of the PCB 1106, chemicals emitted from the battery 1104 may readily pass into the opening of the chemical sensor 1108 for detection.

The chemical sensor 1108 may also be located relatively close to the battery 1104 inside the housing 1102. For example, the chemical sensor may be disposed within 0.25 cm of the battery 1104, within 0.50 cm, within 0.75 cm, within 1.0 cm, within 2.5 cm, within 3 cm, within 5 cm, within 7.5 cm, and/or within 10 cm of the battery 1104, depending on the arrangement of internal components of the thermostat 1100 in various embodiments. Some implementations may use the chemical sensor 1108 to exclusively monitor conditions within the thermostat 1100, and may thus be isolated from the environment external to the thermostat 1100. In these implementations, the chemical sensor 1108 may be located away from an edge of the thermostat and away from the housing 1102.

FIG. 11B illustrates a side view of the thermostat 1100 where the chemical sensor 1108 may also be exposed to an external environment 1112 of the thermostat 1100, according some embodiments. In addition to detecting volatile chemicals that may be emitted from the battery 1104, some embodiments may also use the chemical sensor 1108 to detect volatile chemicals that originate outside of the thermostat 1100. The processor receiving the readings from the chemical sensor 1108 may then classify the measurements received from the chemical sensor 1108 in order to distinguish between chemicals originating from the battery versus chemicals originating from outside the thermostat 1100.

As illustrated in the figures above, the housing 1102 may include multiple sections, such as a sidewall 208 that mates with the ring 210 in FIG. 7. The sections of the housing 1102 may leave a gap 1110 that may also expose the chemical sensor 1108 to chemicals originating in the external environment 1112 outside of the thermostat 1100. As used herein, the internal environment 1114 inside the thermostat 1100 may be defined as inside of a volume formed by the sections of the housing 1102. Conversely, the external environment 1112 outside the thermostat 1100 may be defined as outside of the volume formed by the sections of the housing 1102. Note that the internal environment 1114 inside of thermostat 1100 need not be hermetically or otherwise sealed from the external environment 1112 outside of the thermostat 1100, and the gap 1110 may allow air pass between these two environments.

In order to detect chemicals originating in the external environment 1112 outside of the thermostat 1100, the chemical sensor 1108 may be positioned on the PCB 1106 such that the chemical sensor 1108 is in proximity to both the battery 1104 and the gap 1110. Therefore, the chemical sensor 1108 may be mounted to an outside edge of the PCB 1106. The chemical sensor 1108 may also be mounted within 0.25 cm of the gap 1110, within 0.50 cm, within 0.75 cm, within 1.0 cm, within 2.5 cm, within 3 cm, within 5 cm, within 7.5 cm, and/or within 10 cm of the gap 1110, depending on the arrangement of internal components of the thermostat 1100 in various embodiments. This arrangement allows the chemical sensor 1108 to react quickly to both internal chemicals and external chemicals. Although the gap 1110 may be relatively small (e.g., less than 1 mm, less than 2 mm, less than 3 mm, less than 4 mm, or less than 5 mm, depending on the embodiment), this may allow sufficient airflow through the internal environment 1114 of the thermostat to result in a fast response time to chemicals originating in the external environment 1112 outside of the thermostat 1100.

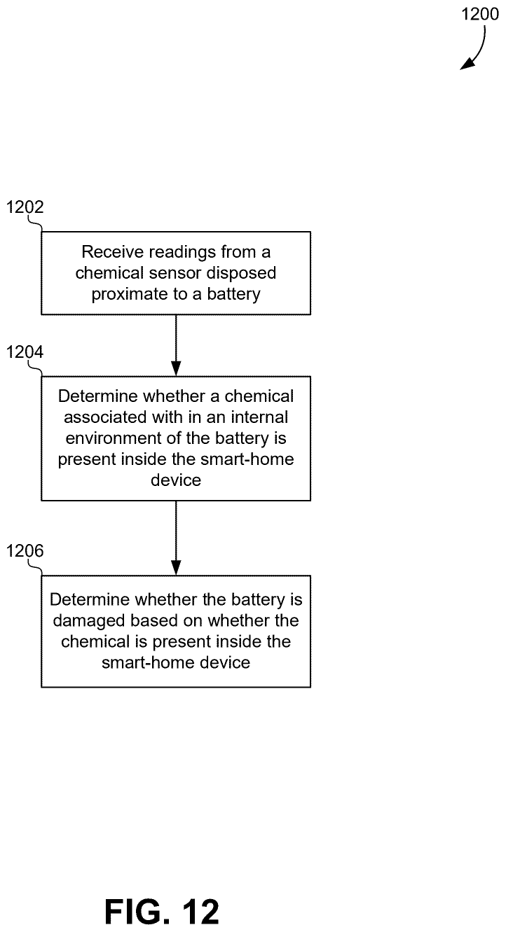

FIG. 12 illustrates a flowchart of a method 1200 for detecting battery anomalies, according to some embodiments. This method may executed by any type of processor. For example, as described above, the smart thermostat may include one or more microprocessors that control operations of the thermostat. The processor(s) may be used in conjunction with the chemical sensor described above to monitor for the presence of volatile chemicals originating inside and/or outside of the thermostat. The processor(s) may also be configured to interact with the HVAC system, the remote server, the local smart-home network, other devices in the smart home environment, and/or software applications operating on these devices. Note that these operations may be performed by one or more processors in a distributed fashion. For example, some operations may be performed by the thermostat processor(s), while other operations are performed by processors at a server or another smart home device in the home. Alternatively, operations may be split between multiple processors within the thermostat. These operations may be embodied in processor instructions that are stored on storage devices. For example, one or more memory devices (e.g., non-transitory computer-readable media) may store instructions that, when executed by the one or more processors, cause the one or more processors to perform the operations described below.

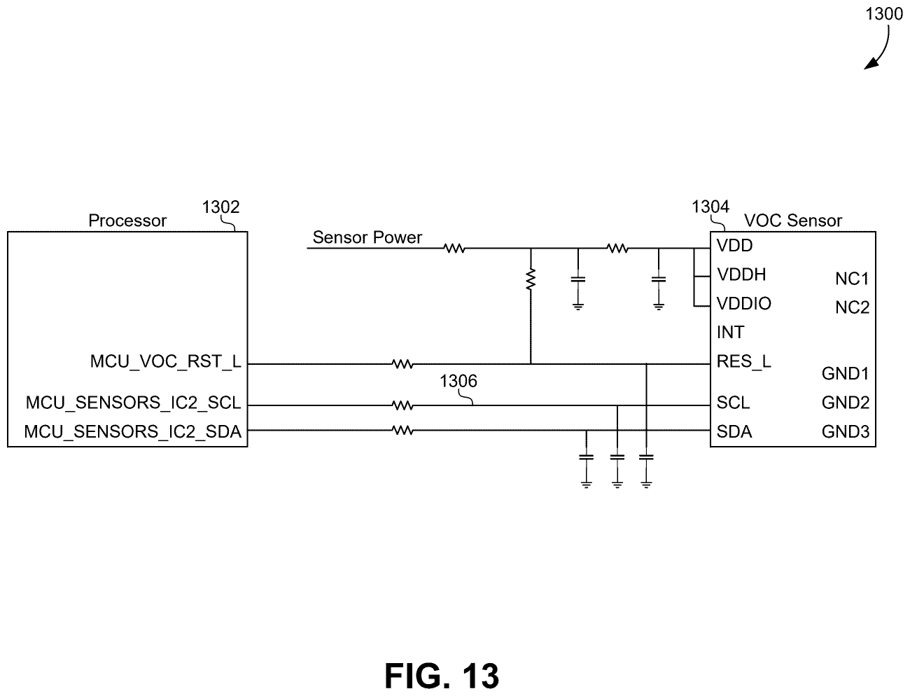

The method may include receiving readings from a chemical sensor disposed proximate to a battery within a housing of a smart-home device (1202). FIG. 13 illustrates a processor 1302 in communication with a chemical sensor 1304, according to some embodiments. In this embodiment, the chemical sensor may be implemented using a Total VOC (TVOC) sensor, such as the ZMOD4410 gas sensor module from Renesas®. Note that this particular chemical sensor is provided only by way of example and is not meant to be limiting. Any other chemical sensor may also be used in different embodiments.

The chemical sensor 1304 may be configured to detect a total amount of volatile organic compounds in the surrounding environment. As described above, the chemical sensor 1304 may include an opening in the top of the housing of the chemical sensor 1304 that exposes a chemical resistance circuit to the surrounding environment. The chemical sensor 1304 may also include software libraries or algorithms that process the chemical resistance measurements in real-time to provide characterizations of the surrounding air quality. For example, some chemical sensors may provide libraries that estimate particular gases (e.g., CO₂), output a total concentration of VOCs, output a reading of the indoor air quality, and so forth. However, these onboard algorithms are not sufficient to detect chemicals emanating from a damaged battery and distinguish those chemicals from others that may be present external to the thermostat.

In order to distinguish between internal battery chemicals and external chemicals, some embodiments may use the processor 1302 on the thermostat to process the raw resistance measurements from the chemical sensor 1304. For example, the chemical sensor 1304 may output a plurality of “pixels” of resistance values for each measurement. The processor may continuously sample these resistance values from the chemical sensor 1304. For example, a set of readings from the chemical sensor 1304 may be sampled every 1 second, every 2 seconds, every 3 seconds, every 4 seconds, every 5 seconds, every 7 seconds, every 10 seconds, every 15 seconds, every 20 seconds, and/or every 30 seconds. The sample period may be between 1 seconds and 5 seconds, between 1 seconds and 10 seconds, between 1 seconds and 15 seconds, and so forth. The processor 1302 may communicate with the chemical sensor 1304 through a serial communication interface 1306. Any communication interface may use any serial protocol, such as I²C, USB, and so forth.

Turning back to FIG. 12, the method may also include processing the readings from the chemical sensor to determine whether a chemical associated with an internal environment of the battery is present inside the smart-home device (1204). For example, chemicals associated with the internal environment of the battery may include any electrolytes, such as EC(C3H4O3), PC(C4H6O3), DEC(C5H10O3), DMC(C3H6O3), and/or EMC(C4H8O3). The internal environment of the battery may also include graphite, lithium cobalt, lithium hexafluorophosphate, diethyl carbonate, propylene carbonate, ethylene carbonate, copper, aluminum, nickel, and so forth. These chemicals associated with the internal environment of the battery may be contrasted with other chemicals that would be associated with an external environment of the battery and/or the thermostat. For example, these external chemicals may include polyurethane, cleaning chemicals, isopropyl alcohol, carbon monoxide, carbon dioxide, and so forth.

To distinguish between these chemicals, some embodiments may develop a chemical sensor reference or baseline relative to background VOC levels and expectations within a home. Various home VOCs and expected concentrations may be used to create a database of VOC exposures that the device may experience in typical operating conditions within a home. The device may therefore have the ability to “learn” these various conditions and exposures over time when installed within many homes. For example, the central server may collect data from a plurality of thermostats installed in different homes where “normal” VOC levels are reported. These readings may be aggregated to develop a baseline set of readings that may be used to establish a normal range of chemical levels that do not require a response from the thermostat or the smart home system.

This monitoring of background or baseline ranges may establish patterns and VOC concentration levels and timings of how VOCs rise and fall within a home based on airflow and ventilation capabilities. For example, when abnormal VOC levels are detected, the system can monitor how the VOC levels respond to different remedial operations. This allows users to understand how to effectively take advantage of their thermostat VOC sensor, as well as gain confidence that their internal lithium-ion polymer battery is not experiencing any electrolyte leakage. For example, if a cleaning agent for hardwood floors is detected, and the household fan is activated in order to disperse the VOCs, the chemical sensor may monitor how these levels decrease in response to the fan. This allows the smart-home system to immediately identify when the fan activation is not dispersing the VOCs as expected.

In addition to using baseline readings to detect VOCs occurring in the environment external to the thermostat, the processor may also identify VOC levels that may be associated with internal chemicals of the battery, such as an electrolyte leak within the thermostat, including various combination of ethylene carbonate, propylene carbonate, diethyl carbonate, dimethyl carbonate, ethyl methyl carbonate, as well as traces of salt components, such as lithium hexafluorophosphate, pentafluorophosphate, and so forth. Depending on the degree of a pouch breach and/or the size of battery (i.e., the electrolyte weight), reference or calibration tables may be populated with VOC ratings indicating such an event. For example, typical VOC levels for the thermostat battery may be recorded when a puncture is present in the battery housing or pouch. These readings may be established using bench-top test procedures and may be validated when actual battery failures occur in installed thermostats. Thresholds may be derived and correlated between internal device VOC sensors and other data points described below. By combining the VOC sensor readings, the determinations made by the thermostat processor, and the response to remedial actions taken by the smart-home system in response, false positives may be eliminated and a determination of the VOC type may be made with high confidence.

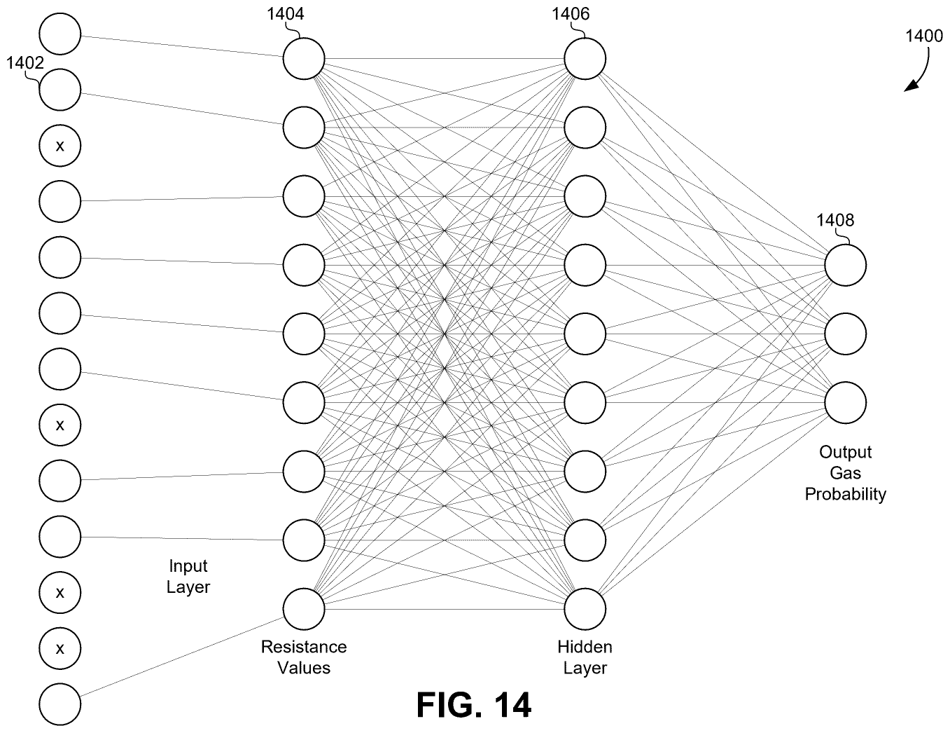

Some embodiments may train artificial-intelligence (AI) or machine-learning (ML) models that may be used to classify detected VOCs as either originating from the battery or from the environment external to the thermostat. FIG. 14 illustrates a diagram of a neural network 1400 used to distinguish between VOCs, according to some embodiments. The neural network 1400 may be implemented on the processor of the thermostat and/or on another processor on another device in the smart-home environment. The neural network 1400 is provided only by way of example and is not meant to be limiting. Other types of models may also be trained and used in other embodiments.

As described above, the outputs from the chemical sensor may include multiple resistance readings for each measurement. In one example, the chemical sensor may provide as many as 13 individual resistance measurements that characterize the VOCs present in the atmosphere around the chemical sensor at a time. However, in order to simplify the calculations and decrease the complexity of the neural network, some embodiments may reduce the number of the resistance measurements by eliminating measurements that may be characterized as having a high level of noise. A noise threshold may be selected, and the plurality of readings received from the chemical sensor may be reduced to remove chemical resistance readings with above the threshold level of noise. In the example of FIG. 14, four of the 13 inputs 1402 may be omitted from being considered inputs of the neural network 1400. The input layer 1404 of the neural network 1400 may thus be reduced down to nine inputs. The neural network may then include at least one hidden layer 1406 that may also include nine nodes.

Turning back briefly to FIG. 12, the method may further include determining whether the battery is damaged based on whether the chemical is present inside the smart-home device (1206). After the hidden internal layers are processed in the neural network 1400, an output layer 1408 may provide a probability for individual gases that may be present. For example, each node in the output layer 1408 may produce a probability between 0.0 and 1.0 indicating the likelihood that the gas corresponding to that output node is present. In this example, there are three output nodes in the output layer 1408, and each of these output nodes may provide a individual probability related to one of three specific gases. These gases may specifically correspond to VOCs that may be present as electrolytes when the battery is damaged. Note that other embodiments may use more or fewer than three output nodes in the output layer 1408 as needed. The probabilities produced by these output nodes may be used to characterize whether a VOC response from the chemical sensor may be attributed to a battery fault or to an external VOC. For example, if the readings from the chemical sensor indicate the presence of a VOC, and the nodes in the output layer 1408 do not exceed a probability threshold, then it may be determined that the VOC is from an external source. Conversely, if the readings of the chemical sensor indicate the presence of a VOC, and at least one of the nodes in the output layer 1408 exceeds a probability threshold, then it may be determined that the VOC may be from a damaged battery.

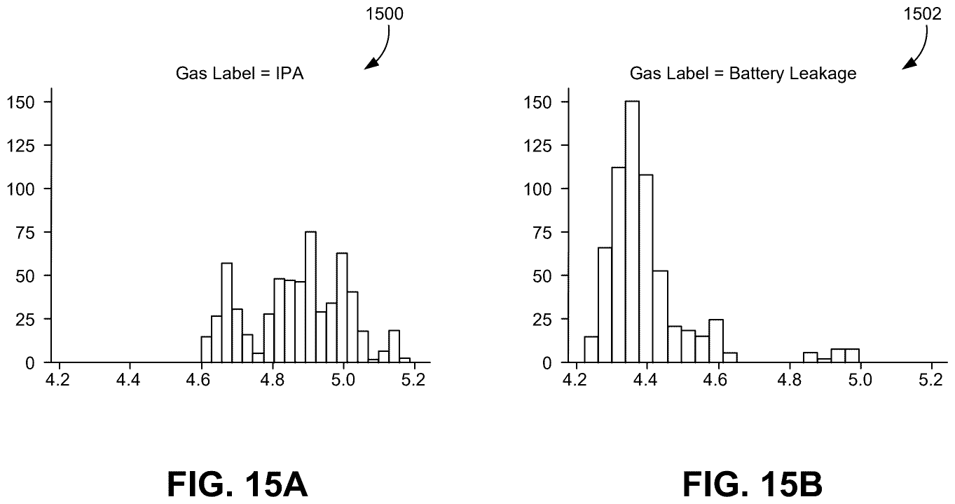

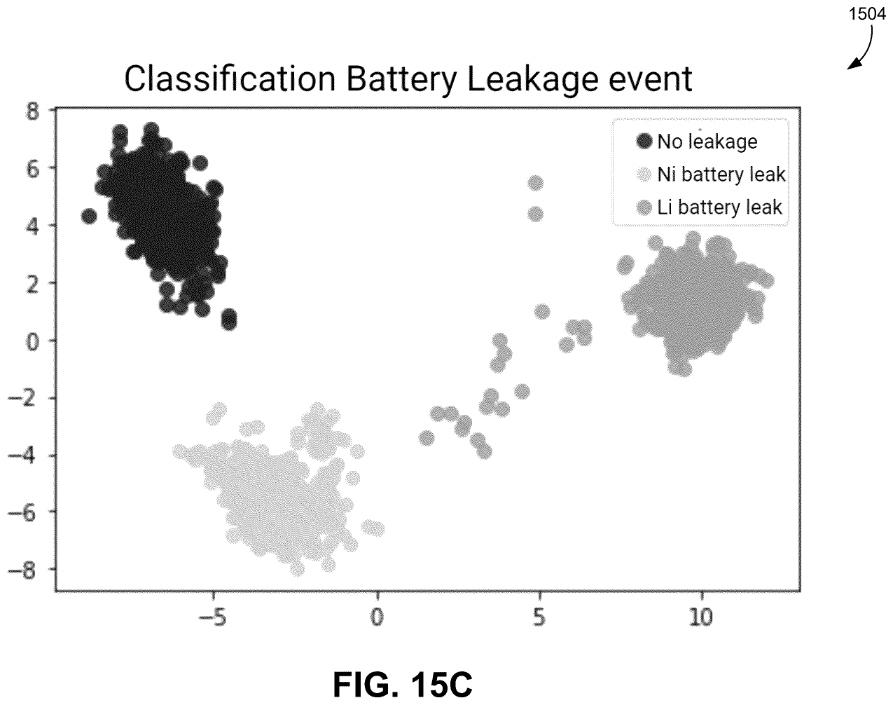

FIG. 15A illustrates a graph 1500 of a response of the chemical sensor for an external VOC, according to some embodiments. FIG. 15B illustrates a graph 1502 of the response of the chemical sensor for a VOC from a leaking battery. Note that the responses between graph 1500 and graph 1502 diverge sufficiently that they can be distinguished from each other using the neural network described above. Experimentally, the neural network 1400 has been shown to distinguish between battery and non-battery VOCs with an accuracy exceeding 99%.

FIG. 15C illustrates a graph 1504 of responses for different gases that may be present from a defective battery, according to some embodiments. Not only is the neural network 1400 able to distinguish between battery and non-battery VOCs with a high level of confidence, but the neural network 1400 is also able to distinguish between different types of VOCs that may be present in different batteries. The graph 1504 illustrates how the responses of the chemical sensor may be clustered tightly together based on different battery types. Cluster 1510 corresponds to no battery leakage, cluster 1512 corresponds to a Ni battery leakage (e.g., a nickel metal hydride (NiMH) battery), and cluster 1514 corresponds to a lithium battery leakage. These clusters are sufficiently distinct for the neural network 1400 to very accurately identify the type of battery-related VOC that may be present in the device. This allows the neural network 1400 to have individual outputs that classify specific chemical compounds that may be associated with the battery. Although not shown explicitly in FIG. 14, some embodiments of the neural network 1400 may also include specific outputs that may be classified as being associated with an environment external to the smart home device.

Some embodiments may perform further processing on the sensor readings and/or the probabilities generated by the neural network in order to improve the performance of the VOC detection process. For example, some embodiments may use a sliding window of a plurality of readings received over time rather than using individual measurement as a basis for a response. The length of the sliding sample window may include any number of samples. For example, some embodiments may use 10-20 samples, 20-40 samples, 40-60 samples, 60-80 samples, 80-100 samples, and so forth. For a window using approximate 100 samples, each successive new sample may be added to the window when the new sample is received, and the oldest sample (i.e., sample 100) may be removed from the sample window.

The processor may then generate a statistic that characterizes or summarizes the plurality of readings over the time interval of the sliding window. The statistic for each input may include median values or average the values for each of the sensor readings over the sliding window. Other summarizing statistics may also be used that may summarize the readings over the time interval of the sliding window different ways. In the example of FIG. 14, a single average value for each of the nine resistance measurements may be calculated from the sliding window and provided as a single input to the neural network 1400. This effectively reduce the effect of transient measurements, momentary spikes, and even momentary detections of VOCs. However, if the sample rate is high enough (e.g., every 1 second to every 5 seconds), the overall response time of the thermostat to a detected VOC will still be very high. For example, the response time may be less than one minute to detect a VOC and begin taking actions to remediate.

Some embodiments may also employ a low-pass filter at various stages of processing the measurements from the chemical sensor. For example, a low-pass filter may be applied over the time interval of the sliding window that smooths each of the individual resistance measurements before they are averaged or otherwise summarized. Alternatively, a low-pass filter may be applied over a time window of probability measurements on the output of the neural network.

The neural network 1400 may be trained using known chemicals that may be present in the home and/or in batteries used by the thermostat. As described above, baseline measurements may be recorded over time for each of these different chemicals, and the corresponding chemical sensor readings may be provided to the neural network as labeled training data when those chemicals are present. The training data may be collected using thermostats installed in user's homes over time. Additionally or alternatively, the training data may be collected in a lab environment where thermostats are exposed to these various chemicals.

Although a single chemical sensor has been disclosed above and shown to effectively detect and distinguish between battery VOCs an external VOCs, other embodiments may use a plurality of chemical sensors to generate a fingerprint pattern of data points that may be associated with each type of VOC. For example, a number of different chemical sensors using different detection techniques (e.g., using different chemical resistors, different detection techniques, different output types, and so forth) may be used in a single device or multiple devices. Each of the sensors may provide different characterizations of a particular VOC, and may use corresponding neural networks trained using those sensor outputs. Alternatively, some embodiments may add VOC sensors to a number of different smart-home devices within the home. For example, a hazard detector, a thermostat, a security camera, a keypad, a smart plug, etc., may include chemical sensors that independently detect a VOC and provide a probability of its presence. Some embodiments may avoid false positives by checking for a corresponding result from a chemical sensor in a neighboring smart-home device. For example, if a thermostat detects a VOC and a nearby hazard detector does not detect the same VOC, this increases the probability that the VOC is leaking from a battery internal to the thermostat.

In addition to initially detecting the presence of a VOC, some embodiments may further take actions to mitigate the VOC. Using the processes described above, a smart-home device may use a VOC sensor to determine that a VOC is present. A processor on smart-home device or otherwise present in the smart-home network may process readings from the chemical sensor to identify a type of the VOC present, and that type may indicate whether the VOC likely originated inside the housing of the smart-home device or outside of the housing of the smart-home device. A sequence of one or more mitigation actions may then be performed by various devices in the smart-home network in response to determining the type of the VOC. For example, there may be a plurality of smart-home devices present in a building/home, and these devices may be communicatively connected through at least one wireless network (e.g., Wi-Fi, Thread, cellular, etc.). The coordination between these multiple devices may help eliminate false positives and increase the confidence in classifying the VOC. Coordinating multiple devices may also allow for more advanced mitigation techniques to be used to remove the VOCs from the environment.

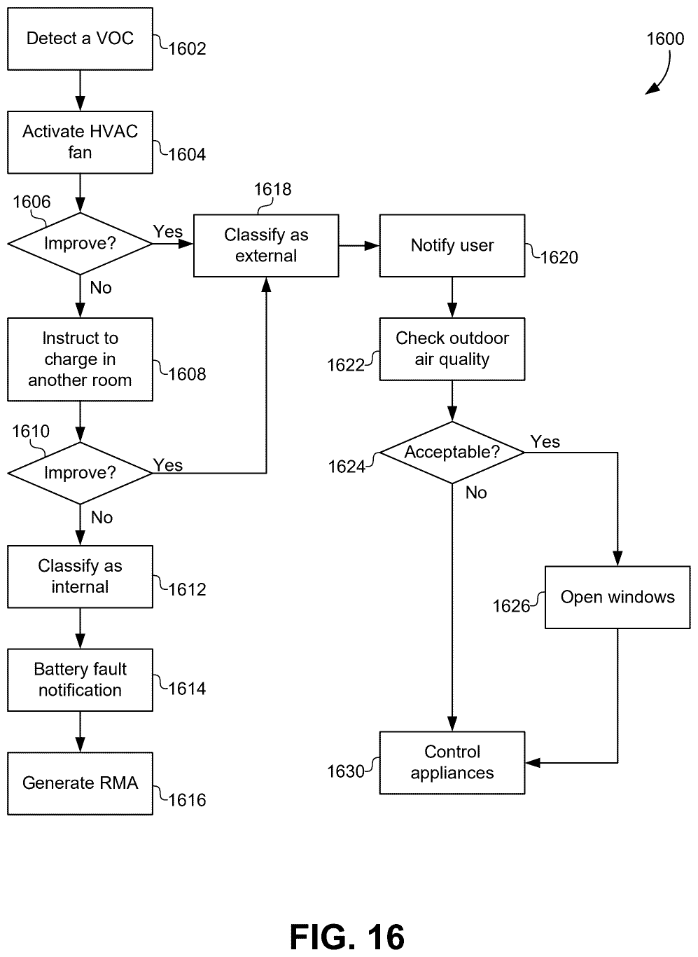

FIG. 16 illustrates a flowchart of various operations that may be performed by the smart-home system to confirm and/or mitigate the presence of the VOC, according to some embodiments. This method may be carried out by a single smart-home device, or by a combination of processors on a plurality of smart-home devices. The method may begin when a VOC is detected (1602). Initially, the VOC may be classified using the chemical sensor, processor, neural network, and other techniques described above. This classification may identify a type of VOC present, such as a specific chemical from a battery or one recognized as an external chemical. As described above, a high level of confidence may already exist when the neural network is fully trained. However, additional mitigation and/or classification steps may be taken even after classification by the neural network to increase the confidence in the classification and remove the VOC from the environment if possible.

After detecting the VOC, an initial step may activate a fan of the HVAC system (1604). For example, the fan or blower function of the HVAC may operate without necessarily providing a heating or cooling function. The fan may be allowed to operate for a few minutes (e.g., one minute, three minutes, five minutes, seven minutes, 10 minutes, etc.) and the chemical sensor readings may be monitored during this time. If the chemical sensor indicates that the air quality around the chemical sensor has not improved (1606) that it may be more likely that a faulty battery may be responsible for the VOC. Some embodiments may then instruct the user to remove the smart-home device from the installed location and take the device into another room for charging or powering through a power port (1608). For example, a notice may be provided on a user interface of the device, on a smart-phone app, or on a digital home assistant instructing the user to remove the device detecting the VOC from its installed location. The instructions may cause the user to take the VOC into a separate room where an external VOC would be less likely to be present and charge plug the VOC into another power source.

After moving the device into another room, the chemical sensor may continue to monitor for the presence of the VOC. If the air quality around the chemical sensor has still not improved (1610), then the classification as a battery-related VOC may be confirmed (1612). Additional mitigation actions may then include providing a notification to a user through one of the available user interfaces that a battery failure has possibly occurred (1614). Some embodiments may even generate a Return Merchandise Authorization (RMA) that may be transmitted to a manufacturer of the device automatically. This may result in instructions to the user to return the device for service or replacement.

If activating the HVAC fan (1604) does show improvement (1606) in the air quality surrounding the chemical sensor, the VOC may be classified as an external VOC (1618). Alternatively, if charging or powering the device in a separate room (1608) shows improvement (1610) in the VOC detection, this may also cause the VOC to be classified as an external VOC (1618). At this stage, additional mitigation actions may be initiated by the smart-home system. For example, warnings or other notifications may be provided to one of the user interfaces for the user (1620). For dangerous levels, the user may be instructed to leave the home. Some embodiments may contact a web service or weather forecast to obtain information regarding the outdoor air quality level (1622). If the outdoor air quality is acceptable or otherwise below a threshold level of pollution, allergens, etc. (1624), then the system may instruct the user to open windows (1626) to provide further ventilation. In homes with automatic windows that may be controlled by the smart home system, the smart home system may open these windows without requiring a user to do so manually.

Further mitigation actions may include controlling other appliances 1630 in the smart home environment. For example a smart plug may be controlled to activate a portable fan. The system may activate an air purification system or other air filters in the home. Some homes may be equipped with a whole-house fan or attic fan that may pull in fresh air from outside to remove the VOC from the environment.

Note that these additional mitigation actions illustrated in FIG. 16 need not be carried out in any particular order. For example, the system may first instruct the user to charge or power the device in another room before activating the HVAC fan. Some embodiments may also include additional mitigation actions that are not explicitly shown in FIG. 16. Furthermore, although a thermostat is used as an example smart-home device in this disclosure, no embodiment need be limited to a thermostat. For example, the chemical sensor may be similarly installed and used by a hazard detector, a security camera, a keypad, a digital home assistant, an intercom, a baby monitor, and/or any other smart-home device.

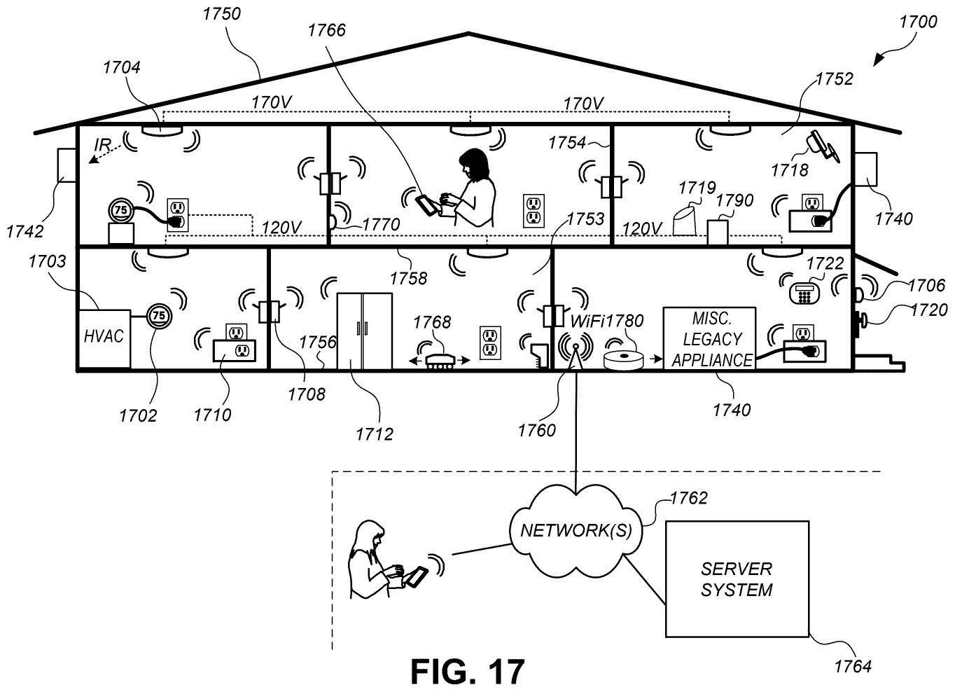

FIG. 17 illustrates an example smart home environment 1700. As shown in FIG. 17, the smart home environment 1700 includes a structure 1750 (e.g., a house, daycare, office building, apartment, condominium, garage, or mobile home) with various integrated devices. It will be appreciated that devices may also be integrated into a smart home environment 1700 that does not include an entire structure 1750, such as an apartment, condominium or office space. Further, the smart home environment 1700 may control and/or be coupled to devices outside of the actual structure 1750. Indeed, several devices in the smart home environment 1700 need not be physically within the structure 1750 (e.g., although not shown, a pool heater, an irrigation system, and the like).

The term “smart home environment” may refer to smart environments for homes such as a single-family house, but the scope of the present teachings is not so limited. The present teachings are also applicable, without limitation, to duplexes, townhomes, multi-unit apartment buildings, hotels, retail stores, office buildings, industrial buildings, and more generally any living space or workspace. Similarly, while the terms user, customer, installer, homeowner, occupant, guest, tenant, landlord, repair person, and the like may be used to refer to the person or persons acting in the context of some particular situations described herein, these references do not limit the scope of the present teachings with respect to the person or persons who are performing such actions. Thus, for example, the terms user, customer, purchaser, installer, subscriber, and homeowner may often refer to the same person in the case of a single-family residential dwelling, because the head of the household is often the person who makes the purchasing decision, buys the unit, and installs and configures the unit, and is also one of the users of the unit. However, in other scenarios, such as a landlord-tenant environment, the customer may be the landlord with respect to purchasing the unit, the installer may be a local apartment supervisor, a first user may be the tenant, and a second user may again be the landlord with respect to remote control functionality. While the identity of the person performing the action may be germane to a particular advantage provided by one or more of the implementations, such identity should not be construed in the descriptions that follow as necessarily limiting the scope of the present teachings to those particular individuals having those particular identities.

The depicted structure 1750 includes a plurality of rooms 1752, separated at least partly from each other via walls 1754. The walls 1754 may include interior walls or exterior walls. Each room may further include a floor 1756 and a ceiling 1758. Devices may be mounted on, integrated with and/or supported by a wall 1754, floor 1756, or ceiling 1758.

In some implementations, the integrated devices of the smart home environment 1700 include intelligent, multi-sensing, network-connected devices that integrate seamlessly with each other in a smart home network and/or with a central server or a cloud-computing system to provide a variety of useful smart home functions. The smart home environment 1700 may include, among other things, one or more intelligent, multi-sensing, network-connected thermostats 1702 (hereinafter referred to as “smart thermostats 1702”), hazard detection units 1704 (hereinafter referred to as “smart hazard detectors 1704”), entryway interface devices 1706 and 1720, and alarm systems 1722 (hereinafter referred to as “smart alarm systems 1722”).

A smart thermostat may detect ambient climate characteristics (e.g., temperature and/or humidity) and control an HVAC system 1703 accordingly. For example, a respective smart thermostat includes an ambient temperature sensor. In some implementations, a respective smart thermostat also includes one or more sensors (e.g., an ambient light sensor and/or a radar sensor) that may be used to control an operation of the respective smart thermostat. For example, based on radar data acquired from a radar sensor included in the smart thermostat and an ambient light level measure by an ambient light sensor included in the smart thermostat, as described above, a display of the smart thermostat may be controlled.

A smart hazard detector may detect smoke, carbon monoxide, and/or some other hazard present in the environment. The one or more smart hazard detectors 1704 may include thermal radiation sensors directed at respective heat sources (e.g., a stove, oven, other appliances, a fireplace, etc.). For example, a smart hazard detector 1704 in a kitchen 1753 includes a thermal radiation sensor directed at a network-connected appliance 1712. A thermal radiation sensor may determine the temperature of the respective heat source (or a portion thereof) at which it is directed and may provide corresponding black-body radiation data as output.

The smart doorbell 1706 and/or the smart door lock 1720 may detect a person's approach to or departure from a location (e.g., an outer door), control doorbell/door locking functionality (e.g., receive user inputs from a portable electronic device 1766 to actuate the bolt of the smart door lock 1720), announce a person's approach or departure via audio or visual means, and/or control settings on a security system (e.g., to activate or deactivate the security system when occupants go and come). In some implementations, the smart doorbell 1706 includes a camera, and, therefore, is also called “doorbell camera 1706” in this document.

The smart alarm system 1722 may detect the presence of an individual within close proximity (e.g., using built-in IR sensors), sound an alarm (e.g., through a built-in speaker, or by sending commands to one or more external speakers), and send notifications to entities or users within/outside of the smart home environment 1700. In some implementations, the smart alarm system 1722 also includes one or more input devices or sensors (e.g., keypad, biometric scanner, NFC transceiver, microphone) for verifying the identity of a user, and one or more output devices (e.g., display, speaker). In some implementations, the smart alarm system 1722 may also be set to an armed mode, such that detection of a trigger condition or event causes the alarm to be sounded unless a disarming action is performed.

In some implementations, the smart home environment 1700 includes one or more intelligent, multi-sensing, network-connected wall switches 1708 (hereinafter referred to as “smart wall switches 1708”), along with one or more intelligent, multi-sensing, network-connected wall plug interfaces 1710 (hereinafter referred to as “smart wall plugs 1710”). The smart wall switches 1708 may detect ambient lighting conditions, detect room-occupancy states, and control a power and/or dim state of one or more lights. In some instances, smart wall switches 1708 may also control a power state or speed of a fan, such as a ceiling fan. The smart wall plugs 1710 may detect occupancy of a room or enclosure and control the supply of power to one or more wall plugs (e.g., such that power is not supplied to the plug if nobody is at home).

In some implementations, the smart home environment 1700 of FIG. 17 includes a plurality of intelligent, multi-sensing, network-connected appliances 1712 (hereinafter referred to as “smart appliances 1712”), such as refrigerators, stoves, ovens, televisions, washers, dryers, lights, stereos, intercom systems, wall clock, garage-door openers, floor fans, ceiling fans, wall air conditioners, pool heaters, irrigation systems, security systems, space heaters, window AC units, motorized duct vents, and so forth. In some implementations, when plugged in, an appliance may announce itself to the smart home network, such as by indicating what type of appliance it is, and it may automatically integrate with the controls of the smart home. Such communication by the appliance to the smart home may be facilitated by either a wired or wireless communication protocol. The smart home may also include a variety of non-communicating legacy appliances 1740, such as old conventional washer/dryers, refrigerators, and the like, which may be controlled by smart wall plugs 1710. The smart home environment 1700 may further include a variety of partially communicating legacy appliances 1742, such as infrared (“IR”) controlled wall air conditioners or other IR-controlled devices, which may be controlled by IR signals provided by the smart hazard detectors 1704 or the smart wall switches 1708.

In some implementations, the smart home environment 1700 includes one or more network-connected cameras 1718 that are configured to provide video monitoring and security in the smart home environment 1700. Cameras 1718 may be mounted in a location, such as indoors and to a wall or can be moveable and placed on a surface. Various embodiments of cameras 1718 may be installed indoors or outdoors. Cameras 1718 may be used to determine occupancy of the structure 1750 and/or particular rooms 1752 in the structure 1750, and thus may act as occupancy sensors. For example, video captured by the cameras 1718 may be processed to identify the presence of an occupant in the structure 1750 (e.g., in a particular room). Specific individuals may be identified based, for example, on their appearance (e.g., height, face) and/or movement (e.g., their walk/gait). Cameras 1718 may additionally include one or more sensors (e.g., IR sensors, motion detectors), input devices (e.g., microphone for capturing audio), and output devices (e.g., speaker for outputting audio). In some implementations, the cameras 1718 are each configured to operate in a day mode and in a low-light mode (e.g., a night mode). In some implementations, the cameras 1718 each include one or more IR illuminators for providing illumination while the camera is operating in the low-light mode. In some implementations, the cameras 1718 include one or more outdoor cameras. In some implementations, the outdoor cameras include additional features and/or components such as weatherproofing and/or solar ray compensation.

The smart home environment 1700 may additionally or alternatively include one or more other occupancy sensors (e.g., the smart doorbell 1706, smart door locks 1720, touch screens, IR sensors, microphones, ambient light sensors, motion detectors, smart nightlights 1770, etc.). In some implementations, the smart home environment 1700 includes radio-frequency identification (RFID) readers (e.g., in each room or a portion thereof) that determine occupancy based on RFID tags located on or embedded in occupants. For example, RFID readers may be integrated into the smart hazard detectors 1704.

Smart home assistant 1719 may have one or more microphones that continuously listen to an ambient environment. Smart home assistant 1719 may be able to respond to verbal queries posed by a user, possibly preceded by a triggering phrase. Smart home assistant 1719 may stream audio and, possibly, video if a camera is integrated as part of the device, to a cloud-based server system 1764 (which represents an embodiment of cloud-based server system 150 of FIG. 1). Smart home assistant 1719 may be a smart device through which non-auditory discomfort alerts may be output and/or an audio stream from the streaming video camera can be output.

By virtue of network connectivity, one or more of the smart-home devices may further allow a user to interact with the device even if the user is not proximate to the device. For example, a user may communicate with a device using a computer (e.g., a desktop computer, laptop computer, or tablet) or another portable electronic device 1766 (e.g., a mobile phone, such as a smart phone). A webpage or application may be configured to receive communications from the user and control the device based on the communications and/or to present information about the device's operation to the user. For example, the user may view a current set point temperature for a device (e.g., a stove) and adjust it using a computer. The user may be in the structure during this remote communication or outside the structure.

As discussed above, users may control smart devices in the smart home environment 1700 using a network-connected computer or portable electronic device 1766. In some examples, some or all of the occupants (e.g., individuals who live in the home) may register their portable electronic device 1766 with the smart home environment 1700. Such registration may be made at a central server to authenticate the occupant and/or the device as being associated with the home and to give permission to the occupant to use the device to control the smart devices in the home. An occupant may use their registered portable electronic device 1766 to remotely control the smart devices of the home, such as when the occupant is at work or on vacation. The occupant may also use their registered device to control the smart devices when the occupant is actually located inside the home, such as when the occupant is sitting on a couch inside the home. It should be appreciated that instead of or in addition to registering portable electronic devices 1766, the smart home environment 1700 may make inferences about which individuals live in the home and are therefore occupants and which portable electronic devices 1766 are associated with those individuals. As such, the smart home environment may “learn” who is an occupant and permit the portable electronic devices 1766 associated with those individuals to control the smart devices of the home.

In some implementations, in addition to containing processing and sensing capabilities, smart thermostat 1702, smart hazard detector 1704, smart doorbell 1706, smart wall switch 1708, smart wall plug 1710, network-connected appliances 1712, cameras 1718, smart home assistant 1719, smart door lock 1720, and/or smart alarm system 1722 (collectively referred to as “the smart-home devices”) are capable of data communications and information sharing with other smart devices, a central server or cloud-computing system, and/or other devices that are network-connected. Data communications may be carried out using any of a variety of custom or standard wireless protocols (e.g., IEEE 802.15.4, Wi-Fi, Matter, ZigBee, 3LoWPAN, Thread, Z-Wave, Bluetooth Smart, ISA100.5A, WirelessHART, MiWi, etc.) and/or any of a variety of custom or standard wired protocols (e.g., Ethernet, HomePlug, etc.), or any other suitable communication protocol, including communication protocols not yet developed as of the filing date of this document.

In some implementations, the smart devices serve as wireless or wired repeaters. In some implementations, a first one of the smart devices communicates with a second one of the smart devices via a wireless router. The smart devices may further communicate with each other via a connection (e.g., network interface 1760) to a network, such as the Internet. Through the Internet, the smart devices may communicate with a cloud-based server system 1764 (also called a cloud-based server system, central server system, and/or a cloud-computing system herein). Cloud-based server system 1764 may be associated with a manufacturer, support entity, or service provider associated with the smart device(s). In some implementations, a user is able to contact customer support using a smart device itself rather than needing to use other communication means, such as a telephone or Internet-connected computer. In some implementations, software updates are automatically sent from cloud-based server system 1764 to smart devices (e.g., when available, when purchased, or at routine intervals).

In some implementations, the network interface 1760 includes a conventional network device (e.g., a router), and the smart home environment 1700 of FIG. 17 includes a hub device 1780 that is communicatively coupled to the network(s) 1762 directly or via the network interface 1760. The hub device 1780 is further communicatively coupled to one or more of the above intelligent, multi-sensing, network-connected devices (e.g., smart devices of the smart home environment 1700). Each of these smart devices optionally communicates with the hub device 1780 using one or more radio communication networks available at least in the smart home environment 1700 (e.g., Matter, ZigBee, Z-Wave, Insteon, Bluetooth, Wi-Fi and other radio communication networks). In some implementations, the hub device 1780 and devices coupled with/to the hub device can be controlled and/or interacted with via an application running on a smart phone, household controller, laptop, tablet computer, game console or similar electronic device. In some implementations, a user of such a controller application can view the status of the hub device or coupled smart devices, configure the hub device to interoperate with smart devices newly introduced to the home network, commission new smart devices, and adjust or view settings of connected smart devices, etc. In some implementations the hub device extends capabilities of low capability smart devices to match capabilities of the highly capable smart devices of the same type, integrates functionality of multiple different device types—even across different communication protocols—and is configured to streamline adding of new devices and commissioning of the hub device. In some implementations, hub device 1780 further includes a local storage device for storing data related to, or output by, smart devices of smart home environment 1700. In some implementations, the data includes one or more of: video data output by a camera device, metadata output by a smart device, settings information for a smart device, usage logs for a smart device, and the like.

In some implementations, smart home environment 1700 includes a local storage device 1790 for storing data related to, or output by, smart devices of smart home environment 1700. In some implementations, the data includes one or more of: video data output by a camera device (e.g., cameras 1718 or smart doorbell 1706), metadata output by a smart device, settings information for a smart device, usage logs for a smart device, and the like. In some implementations, local storage device 1790 is communicatively coupled to one or more smart devices via a smart home network. In some implementations, local storage device 1790 is selectively coupled to one or more smart devices via a wired and/or wireless communication network. In some implementations, local storage device 1790 is used to store video data when external network conditions are poor. For example, local storage device 1790 is used when an encoding bitrate of cameras 1718 exceeds the available bandwidth of the external network (e.g., network(s) 1762). In some implementations, local storage device 1790 temporarily stores video data from one or more cameras (e.g., cameras 1718) prior to transferring the video data to a server system (e.g., cloud-based server system 1764).

Further included and illustrated in the exemplary smart home environment 1700 of FIG. 17 are service robots 1768, each configured to carry out, in an autonomous manner, any of a variety of household tasks. For some embodiments, the service robots 1768 can be respectively configured to perform floor sweeping, floor washing, etc.

In some embodiments, a service robot may follow a person from room to room and position itself such that the person can be monitored while in the room. The service robot may stop in a location within the room where it will likely be out of the way, but still has a relatively clear field-of-view of the room.

The systems and methods of the present disclosure may be implemented using hardware, software, firmware, or a combination thereof and may be implemented in one or more computer systems or other processing systems. Some embodiments of the present disclosure include a system including a processing system that includes one or more processors. In some embodiments, the system includes a non-transitory computer readable storage medium containing instructions which, when executed on the one or more processors, cause the system and/or the one or more processors to perform part or all of one or more methods and/or part or all of one or more processes disclosed herein. Some embodiments of the present disclosure include a computer-program product tangibly embodied in a non-transitory machine-readable storage medium, including instructions configured to cause the system and/or the one or more processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification, and variation of the concepts herein disclosed may be resorted to by those skilled in the art, and that such modifications and variations are considered to be within the scope of this invention as defined by the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

The above description of certain examples, including illustrated examples, has been presented only for the purpose of illustration and description and is not intended to be exhaustive or to limit the disclosure to the precise forms disclosed. Modifications, adaptations, and uses thereof will be apparent to those skilled in the art without departing from the scope of the disclosure. For instance, any examples described herein can be combined with any other examples.

As used herein, the terms “about” or “approximately” or “substantially” may be interpreted as being within a range that would be expected by one having ordinary skill in the art in light of the specification.

In the foregoing description, for the purposes of explanation, numerous specific details were set forth in order to provide a thorough understanding of various embodiments. It will be apparent, however, that some embodiments may be practiced without some of these specific details. In other instances, well-known structures and devices are shown in block diagram form.

The foregoing description provides exemplary embodiments only, and is not intended to limit the scope, applicability, or configuration of the disclosure. Rather, the foregoing description of various embodiments will provide an enabling disclosure for implementing at least one embodiment. It should be understood that various changes may be made in the function and arrangement of elements without departing from the spirit and scope of some embodiments as set forth in the appended claims.

Specific details are given in the foregoing description to provide a thorough understanding of the embodiments. However, it will be understood that the embodiments may be practiced without these specific details. For example, circuits, systems, networks, processes, and other components may have been shown as components in block diagram form in order not to obscure the embodiments in unnecessary detail. In other instances, well-known circuits, processes, algorithms, structures, and techniques may have been shown without unnecessary detail in order to avoid obscuring the embodiments.

Also, it is noted that individual embodiments may have been described as a process which is depicted as a flowchart, a flow diagram, a data flow diagram, a structure diagram, or a block diagram. Although a flowchart may have described the operations as a sequential process, many of the operations can be performed in parallel or concurrently. In addition, the order of the operations may be re-arranged. A process is terminated when its operations are completed, but could have additional steps not included in a figure. A process may correspond to a method, a function, a procedure, a subroutine, a subprogram, etc. When a process corresponds to a function, its termination can correspond to a return of the function to the calling function or the main function.

The term “computer-readable medium” includes, but is not limited to portable or fixed storage devices, optical storage devices, wireless channels and various other mediums capable of storing, containing, or carrying instruction(s) and/or data. A code segment or machine-executable instructions may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc., may be passed, forwarded, or transmitted via any suitable means including memory sharing, message passing, token passing, network transmission, etc.

Furthermore, embodiments may be implemented by hardware, software, firmware, middleware, microcode, hardware description languages, or any combination thereof. When implemented in software, firmware, middleware or microcode, the program code or code segments to perform the necessary tasks may be stored in a machine readable medium. A processor(s) may perform the necessary tasks.

In the foregoing specification, features are described with reference to specific embodiments thereof, but it should be recognized that not all embodiments are limited thereto. Various features and aspects of some embodiments may be used individually or jointly. Further, embodiments can be utilized in any number of environments and applications beyond those described herein without departing from the broader spirit and scope of the specification. The specification and drawings are, accordingly, to be regarded as illustrative rather than restrictive.

Additionally, for the purposes of illustration, methods were described in a particular order. It should be appreciated that in alternate embodiments, the methods may be performed in a different order than that described. It should also be appreciated that the methods described above may be performed by hardware components or may be embodied in sequences of machine-executable instructions, which may be used to cause a machine, such as a general-purpose or special-purpose processor or logic circuits programmed with the instructions to perform the methods. These machine-executable instructions may be stored on one or more machine readable mediums, such as CD-ROMs or other type of optical disks, floppy diskettes, ROMs, RAMs, EPROMs, EEPROMs, magnetic or optical cards, flash memory, or other types of machine-readable mediums suitable for storing electronic instructions. Alternatively, the methods may be performed by a combination of hardware and software.

What is claimed is:

1. A thermostat comprising:
a housing;
a battery disposed within the housing and exposed to an internal environment of the thermostat; and
a Volatile Organic Compound (VOC) sensor disposed within the housing exposed to the internal environment of the thermostat and the battery.

2. The thermostat of claim 1, wherein the VOC sensor is disposed within 3 cm of the battery.

3. The thermostat of claim 1, wherein the VOC sensor is sealed from an environment that is external to the housing of the thermostat such that the VOC sensor is not exposed to the environment that is external to the housing of the thermostat or to VOCs originating outside of the housing of the thermostat.

4. The thermostat of claim 1, wherein the VOC sensor is also exposed to an environment that is external to the housing of the thermostat such that the VOC sensor is exposed to VOCs originating outside of the housing of the thermostat.

5. The thermostat of claim 1, wherein the VOC sensor is configured to detect an electrolyte that is emitted from the battery when the battery is damaged.

6. The thermostat of claim 5, wherein the battery comprises a lithium-ion battery, and the electrolyte comprises one or more of EC(C3H4O3), PC(C4H6O3), December (C5H10O3), DMC(C3H6O3), and/or EMC(C4H8O3).

7. The thermostat of claim 1, further comprising a processor, wherein the VOC sensor generates measurements from a chemical resistance circuit, and the VOC comprises a serial interface to transmit the chemical resistance circuit to the processor.

8. A method of detecting battery anomalies, the method comprising:
receiving readings from a chemical sensor disposed proximate to a battery within a housing of a smart-home device;
processing the readings from the chemical sensor to determine whether a chemical associated with an internal environment of the battery is present inside the smart-home device; and
determining whether the battery is damaged based on whether the chemical is present inside the smart-home device.

9. The method of claim 8, wherein the readings from the chemical sensor comprise a sliding window of a plurality of readings over a time interval, and determining whether the chemical is present comprises calculating a statistic summarizing the plurality of readings over the time interval.

10. The method of claim 9, further comprising applying a low-pass filter to the plurality of readings over the time interval.

11. The method of claim 9, reducing the plurality of readings to a fewer number of readings to remove chemical resistance readings with above a threshold level of noise.

12. The method of claim 8, wherein the readings comprise chemical resistance measurements from a Volatile Organic Compound (VOC) sensor.

13. The method of claim 8, further comprising providing the readings from the chemical sensor to a neural network, wherein the neural network comprises inputs corresponding to a plurality of the readings, at least one hidden internal layer, and outputs corresponding to specific chemical compounds.

14. The method of claim 13, wherein the outputs provide a probability indicating a likelihood of the corresponding specific chemical compounds being present.

15. The method of claim 13, wherein the outputs classify the specific chemical compounds as being associated with the battery or being associated with an environment external to the smart-home device.

16. The method of claim 8, wherein the readings are received from the chemical sensor with a sampling period of between every 1 second and every 5 seconds.

17. A method of detecting Volatile Organic Compounds (VOCs) in smart-home devices, the method comprising:
determining, using a VOC sensor of a smart-home device, that a VOC is present in a housing of the smart-home device, wherein the smart-home device is one of a plurality of smart-home devices present in a building, and the plurality of smart-home devices are communicatively connected through at least one wireless network in the building;
determining, by a processor, a type of the VOC present in the smart-home device, wherein the type of the VOC indicates whether the VOC likely originated inside the housing of the smart-home device or outside of the housing of the smart-home device; and
causing a sequence of one or more mitigation actions to be performed by the plurality of smart-home devices in response to determining the type of the VOC.

18. The method of claim 17, wherein the sequence of one or more mitigation actions comprises causing a ventilation system of the building to be activated when the type of VOC indicates that the VOC likely originated outside of the housing of the smart-home device.

19. The method of claim 17, wherein the sequence of one or more mitigation actions comprises connecting to a smart-home app or server of a manufacturer of the smart-home device to arrange for a replacement or repair of the smart-home device.

20. The method of claim 17, wherein the sequence of one or more mitigation actions comprises evaluating an outdoor air quality before causing a window of the building to be opened.
